(19) United States
(12) Patent Application Publication
(10) Pub. No.: US 2026/0226121 A1
(43) Pub. Date: Aug. 6, 2026

(54) METHODS OF OBTAINING KERATIN PROTEIN ISOLATE, KERATIN AMYLOID FIBRILS AND FABRICATING MEMBRANES USING THE SAME

(71) Applicants: NANYANG TECHNOLOGICAL UNIVERSITY, Singapore (SG); ETH ZURICH, Zurich (CH)

(72) Inventors: Raffaele MEZZENGA, Volketswll (CH); Wei Long SOON, Singapore (SG); Ali Gilles Tchenguise MISEREZ, Singapore (SG)

(21) Appl. No.: 19/148,568

(22) PCT Filed: Jan. 16, 2024
(86) PCT No.: PCT/SG2024/050036

§ 371 Date: Jul. 16, 2025

(30) Foreign Application Priority Data
Jan. 16, 2023 (SG) 10202300115R

(51) Int. Cl. C07K 14/47; C08L 89/04; H01M 8/1004; H01M 8/1069; H01M 8/1072

(52) U.S. Cl. CPC C07K 14/4741; C08L 89/04; H01M 8/1004; H01M 8/1072; H01M 8/1079

(57) ABSTRACT

The present invention relates to a method of obtaining keratin protein isolate from a keratin source, the method comprising: (a) preparing a first mixture comprising keratin source, chaotropic solvent, and a first reducing agent; (b) precipitating keratin protein from the first mixture; (c) preparing a second mixture comprising the keratin protein and a second reducing agent; and (d) precipitating keratin protein isolate from the second mixture. The present invention also relates to a method of obtaining keratin amyloid fibrils, the method comprising: (i) obtaining keratin protein isolate; (ii) preparing a third mixture comprising keratin protein isolate, acid solution, and a third reducing agent; and (iii) subjecting the third mixture to heat treatment to obtain keratin amyloid fibrils. The present invention also relates to a method of fabricating a membrane for an electrochemical cell or a proton conductive cell.

### TECHNICAL FIELD

The present invention generally relates to extracting keratin proteins from a keratin source, and more particularly relates to obtaining keratin protein isolates and/or keratin amyloid fibrils from a keratin source. The present invention also relates to methods comprising the use of keratin amyloid fibrils in membrane fabrication, more particularly membranes for electrochemical applications or other applications related to proton transport.

### BACKGROUND ART

Over the past few decades, increasing carbon emissions have accelerated climate change to a level where its devastating effects have become tangible on an everyday basis. This is mirrored by a projected increase in global energy demand by about 50% in the span of a single generation, urging a shift from fossil-fuel derived materials (which are non-renewable and pose detrimental environmental issues) towards greener materials and more sustainable manufacturing processes. In order to mitigate global warming and build a sustainable future of reduced emissions of greenhouse gases (such as carbon dioxide (CO₂)), it is desirable to replace traditional combustion of fossil fuels to generate electricity with new greener technologies based on renewable and sustainable energy generation sources.

Fuel cells are considered a promising sustainable technology for energy conversion using electrochemical reactions to generate electricity without CO₂ emissions. Among the various types of fuel cells, hydrogen fuel cells are particularly attractive from a decarbonization point-of-view, with efforts focusing on the production of green hydrogen as the next renewable fuel. The heart of a fuel cell lies in the membrane electrode assembly (MEA), which consists of a proton exchange membrane (PEM) that selectively allows only the passage of protons between the electrodes. For example, hydrogen polymer electrolyte membrane fuel cells (PEMFC), which operate using only hydrogen and air, provide a sustainable avenue due to the production of only water as by-product without producing carbon dioxide. The core of the PEMFC lies in a proton conductive membrane which allows only the transport of protons across.

Such fuel cells use perfluorosulfonic acid (PFSA) membranes such as Nafion® and Aquivion® in fuel cells due to their high proton conductivity, chemical inertness, and mechanical stability. However, the cost of producing such Nafion® membranes is very expensive due to the use of fluorinated chemicals. Furthermore, their manufacturing processes are hazardous and pose serious environmental concerns relating to poor biodegradability and environmental toxicity of perfluorinated materials. Moreover, functionalization of polymers through sulfonation usually involves highly corrosive and hazardous reagents, reducing their environmental friendliness.

Hence, there is a need to provide a solution, such as alternative membranes for electrochemical applications, that overcomes, or at least ameliorates, one or more of the disadvantages described above. Furthermore, other desirable features and characteristics will become apparent from the subsequent detailed description and the appended claims, taken in conjunction with the accompanying figures and this background of the disclosure.

### SUMMARY

In one aspect of the present disclosure, there is provided a method of obtaining keratin protein isolate from a keratin source, the method comprising:
(a) preparing a first mixture comprising keratin source, chaotropic solvent, and a first reducing agent;
(b) precipitating keratin protein from the first mixture;
(c) preparing a second mixture comprising the keratin protein and a second reducing agent; and
(d) precipitating keratin protein isolate from the second mixture.

In another aspect of the present disclosure, there is provided a method of obtaining keratin amyloid fibrils, the method comprising:
(i) obtaining keratin protein isolate as disclosed herein;
(ii) preparing a third mixture comprising keratin protein isolate, acid solution, and a third reducing agent; and
(iii) subjecting the third mixture to heat treatment to obtain keratin amyloid fibrils.

Advantageously, the keratin source may be hair, nails, feathers, claws, beaks, horns, hoofs, and wools from human, birds and/or animals. These are readily available and are usually generated as waste.

These non-valuable waste keratin sources can be reconfigured into valuable products, such as keratin protein isolate and/or keratin amyloid fibrils. This advantageously helps to reduce waste and build a circular economy by turning waste into highly functional materials.

Also advantageously, the disclosed methods may be easy-to-perform, safe and do not pose toxicity issues. The chemicals used in the disclosed methods may be easily obtained, utilize safe (not harsh) reagents, and are low-cost.

In a further aspect of the present disclosure, there is provided a method of fabricating a membrane for an electrochemical cell, the method comprising:
(A) forming a film by combining keratin amyloid fibrils obtained as disclosed herein, a crosslinking agent, and a dopant;
(B) heat treating the film to induce crosslinking; and
(C) subjecting the crosslinked film to oxidation and protonation to form the membrane.

Advantageously, the disclosed method of fabricating a membrane may be a sustainable, green, and cost-effective method. The chemicals may be generally safe, harmless, and do not pose additional environmental issues and pollution to the environment, as compared to the hazardous and toxic fluorinated chemicals typically used in preparing commercial fuel cells and the highly corrosive process of functionalization of polymers typically used in preparing commercial fuel cells. Furthermore, the cost of the materials used in the present disclosure may be generally less expensive as compared to fluorinated chemicals.

Further advantageously, the disclosed method of fabricating a membrane may be simple, straightforward and easy-to-perform. The membrane fabrication method itself may be scalable, non-hazardous, water based, and thus does not introduce additional pollution to the environment. The disclosed method also allows for expansion to large-scale applications.

Also advantageously, the membranes may be fabricated from waste or low-cost keratin sources. The use of waste or low-cost keratin sources enables the reduction of waste and builds a circular economy by reconfiguring waste into highly functional membranes for electrochemical applications (e.g. electrochemical cells) or other applications related to proton transport (e.g. proton conductive cell). Hence, the disclosed method adds value to the circular economy by valorization of a waste product into useful membranes.

### Definitions

Unless otherwise defined herein, scientific and technical terms used in this application shall have the meanings that are commonly understood by those of ordinary skill in the art. Generally, nomenclature used in connection with, and techniques of, chemistry described herein, are those well-known and commonly used in the art.

As used herein, the term “chaotropic agent” refers to an agent that is a molecule in water solution that disrupts the hydrogen bonding network between water molecules (i.e. exerts chaotropic activity). The chaotropic agent disrupts the hydrogen bonding network between water molecules and in turn reduces the stability of the native state of proteins by weakening its hydrophobic effect and causing protein denaturation.

As used herein, the term “keratin amyloid fibrils” refers to keratin protein in the aggregated form of nanofibrils, of widths within nanometer length scale and lengths of micrometer length scales.

As used herein, the term “reducing agent” refers to a chemical species that donates an electron to an electron recipient (called the oxidizing agent, oxidant, oxidizer, or electron acceptor). The reducing agent may be also known as a reductant, reducer, or electron donor.

As used herein, the term “crosslinking agent” is to be interpreted broadly to include any chemicals added or used to form a network of crosslinks through the process of chemical crosslinking, physical linkage, ionic, and/or hydrogen bonding.

As used herein, the term “dopant” refers to a small amount of a substance added to a material to alter its physical properties to facilitate crosslinking or improve the properties of the formed film or membrane.

The word “substantially” does not exclude “completely” e.g. a composition which is “substantially free” from Y may be completely free from Y. Where necessary, the word “substantially” may be omitted from the definition of the invention.

Unless the context requires otherwise or specifically stated to the contrary, integers, steps, or elements of the invention recited herein as singular integers, steps or elements clearly encompass both singular and plural forms of the recited integers, steps or elements.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range.

For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### BRIEF DESCRIPTION OF DRAWINGS

The accompanying drawings illustrate disclosed embodiments and serve to explain the principles of the disclosed embodiments. It is to be understood, however, that the drawings are designed for purposes of illustration only, and not as a definition of the limits of the invention.

FIG. 1 illustrates a method of using poultry industrial waste as a keratin source to obtain keratin protein isolates and keratin amyloid fibrils to ultimately fabricate membranes for fuel cell applications in accordance with an embodiment of the present invention.

FIG. 2 shows an analysis and characterization of a keratin source (feathers) and keratin amyloid fibrils extracted and/or isolated from the keratin source in an embodiment of the present invention; where (a) is a graph showing a comparison of cysteine (Cys) content (%) of the keratin source with several other major proteins from industrial food waste; (b) is a sodium dodecyl-sulfate polyacrylamide gel electrophoresis (SDS-PAGE) of a non-reduced (without a reducing agent) and a reduced (with reducing agent) isolated feather keratin; (c) is a graph showing fibrillization behavior of feather keratin amyloid fibrils with time, as assessed with the thiazole orange (inset); (d) is an image of keratin amyloid fibril solution under cross-polarized light; (e) shows an Atomic Force Microscopy (AFM) image of feather keratin amyloid fibrils and a series of graphs showing AFM statistical analysis of feather keratin amyloid fibrils showing fibril pitch, average fibril height, and contour length; and (f) shows a Transmission Electron Microscopy (TEM) image of feather keratin amyloid fibrils.

FIG. 3 is a pie chart diagram showing the amino acid composition of isolated feather keratin from chicken feathers.

FIG. 4 is a series of sequences and graphs showing the analysis and prediction of amyloidogenic aggregation behaviour of feather keratin from chicken feathers, where (a) and (b) show sequences of feather keratin with parallel aggregation regions in the shaded regions; while (c), (d) and (c) is a series of graphs showing aggregation prediction sites using TANGO. PASTA, and Aggrescan algorithms, respectively; and (f) is a graph showing amyloidogenic sites based on normalized hotspot area (NHSA) using MetAmyl.

FIG. 5 is a series of photographs showing images of a membrane of an embodiment of the present invention (keratin-M membranes) during fabrication: (a) after solvent casting before treatment; (b) after thermal curing (heat treating); (c) after post treatment of oxidation and protonation; and (d) post-treated membranes under cross-polarized light.

FIG. 6 is a series of spectra, graphs and/or images showing the analysis and characterization of various membranes obtained from the present invention, where (a) is a Fourier Transform Infrared Spectroscopy (FTIR) spectra of keratin-M membrane and a pretreated keratin-M membrane (keratin-M membrane before pre-treatment); (b) is a Raman spectra of keratin-M membrane (solid line) and a keratin membrane that was not fabricated with a dopant (neat keratin membranes) (dotted line); (c) is a X-ray photoelectron spectroscopy (XPS) spectrum of the keratin-M membrane; (d) is a graph illustrating the surface potential of the keratin-M membrane before and after treatment using Kelvin probe force microscopy (KPFM); (e) is a X-ray diffraction (XRD) spectrum showing patterns of a keratin-M membrane and a neat keratin membrane; and (f) is a Scanning Electron Microscopy (SEM) image of the keratin-M membrane.

FIG. 7 is a series of X-ray photoelectron spectroscopy (XPS) spectra of keratin-M membranes of the present invention; (a) O 1s; (b) N 1s; (c) C 1s; and (d) S 2p.

FIG. 8 is a series of images and/or graphs showing the analysis and characterization of a keratin-M membrane on highly ordered pyrolytic graphite (HOPG) substrate before treatment and after treatment, where in each image (a) and (b), a photograph in the upper left corner is an image showing the AFM probe on HOPG-Membrane interface, while a photograph in the upper right corner is an image showing the 3D height profile of the interface, and the bottom graph shows the surface potential across interface; (c) and (d) are AFM potential images of keratin-M membrane on HOPG substrate before treatment and after treatment; (e) and (f) are AFM height images of keratin-M membrane on HOPG substrate before treatment and after treatment; and (g) and (h) are AFM amplitude images of keratin-M membrane on HOPG substrate before treatment and after treatment.

FIG. 9 is a series of images showing a cross section of keratin-M membranes of the present invention, where the membranes are fabricated with a dopant (mercaptosuccinic acid (MSA)); (a) surface (with low magnification); and (b) cross section (with high magnification).

FIG. 10 is a series of images showing a cross section of membranes that were not fabricated with a dopant (neat keratin membranes); (a) surface (with low magnification); and (b) cross section (with high magnification).

FIG. 11 is (a) a photograph showing keratin-M membranes of the present invention demonstrating no permeability to large dye molecules such as Rhodamine B (i.e. Rhodamine B rejection of keratin-M membrane) at 0 hour and 24 hours; and (b) an absorbance spectrum of solutions in each chamber.

FIG. 12 is a bar graph showing methanol permeability of a keratin-M membrane (fabricated with a dopant MSA) of the present invention and a neat keratin membrane (not fabricated with a dopant; no MSA) in comparison with Nafion 212.

FIG. 13 is a graph showing tensile test measurement (a graph of stress (MPa) against strain) of a keratin-M membrane of the present invention and a neat keratin membrane (not fabricated with a dopant).

FIG. 14 is a series of graphs showing dynamic mechanical analysis (a) storage modulus against temperature; and (b) tan 0 against temperature of a keratin-M membrane (fabricated with a dopant MSA) of the present invention and a neat keratin membrane (not fabricated with a dopant; no MSA).

FIG. 15 is a graph of weight (%) against temperature (Ts/° C.) showing thermogravimetric analysis of keratin-M membrane of the present invention.

FIG. 16 is a series of graphs and images showing characterization and analysis of keratin membranes (neat keratin membrane and keratin-M membrane) and their performance in fuel cells and transistors, where (a) is a bar graph showing proton conductivities of keratin membranes with increasing dopant (MSA) content; of 0 weight % MSA, 10 weight % MSA and 20 weight % MSA; (b) and (c) are Nyquist plots of neat keratin membrane and keratin-M membrane respectively; (d) and (e) are graphs showing in-situ performance of keratin-M membrane at different temperatures using hydrogen at the anode and (d) air and (e) oxygen at the cathode; (f) is a series of photographs showing the assembly of keratin-M membranes into a commercial fuel cell to power devices such as the powering of a commercial fuel cell toy car as shown; (g) is a schematic illustration showing protonic field-effect transistor (FET) using keratin-M membrane; (h) is a graph showing I_{SD}-V_{SD} plot of keratin-M membrane with increasing relative humidity (I_{SD} in log scale); and (i) is a graph showing a transfer characteristic of keratin-M bioprotonic FET device at 90% RH and V_{SD}=0.5 V.

FIG. 17 is a series of graphs showing Bode plots of (a) neat keratin; (b) keratin-M.

FIG. 18 is a series of images showing (a) keratin-M membrane of the present invention used as a polymer electrolyte membrane in an in-situ hydrogen fuel cell setup; and (b) in-situ measurement setup.

FIG. 19 is a series of graphs showing an in-situ performance of keratin-M membranes at 65° C., followed by 80° C., and cooling down to 65° C. when using (a) air as cathode and (b) oxygen as cathode.

FIG. 20 is a graph of current density (mA/cm²) against time (s) showing staircase voltammetry of hydrogen crossover experiment (hydrogen permeability) of keratin-M membrane.

FIG. 21 is a graph showing current density profile with voltage for hydrogen crossover assessment (hydrogen permeability) of keratin-M membrane.

FIG. 22 is a series of images showing a Keratin-M fuel cell device powering of (a) a white light-emitting diode (LED) lamp and (b) a red LED lamp.

FIG. 23 is a series of photographs showing electrolysis with keratin-M membrane of the present invention, where left-hand-side of each photograph shows hydrogen (H₂) production and right-hand-side of each photograph shows water (H₂O) and oxygen (O₂) production where the black arrows indicate bubble formation at the respective outlets.

FIG. 24 is a graph showing I_{SD}-V_{SD} behaviour of keratin-M membrane transistors from negative to positive gate voltage.

### DETAILED DISCLOSURE OF EMBODIMENTS

The following detailed description is merely exemplary in nature and is not intended to limit the invention or the application and uses of the invention. Furthermore, there is no intention to be bound by any theory presented in the preceding background of the invention or the following detailed description. It is the intent of the present invention to present a membrane for electrochemical applications that overcomes, or at least ameliorates, one or more of the disadvantages described earlier in the background of the disclosure.

The ongoing research of fuel cell membranes generally focuses on the improvement of polymeric materials due to easy functionalization and tunable repetitive units. Although these polymeric materials possess high proton conductivity, they are petroleum-based, which increases the environmental impact and reduces the sustainability factor due to limited resources, while lacking biodegradability.

Naturally occurring bio-based materials provide an alternative avenue due to their renewability, abundance, and biodegradability. Bio-based materials from industrial by-products, such as food industry side-streams, are attractive alternatives with a strong potential for valorisation owing to their large volume generation, low cost, renewability and biodegradability, and intrinsic material properties. For example, chicken feathers am generated at a staggering rate of 40 million tons annually as a by-product from the poultry industry, but have been underutilized due to their low nutritional profile. Furthermore, disposing of chicken feathers face challenges due to the generation of toxic sulphur dioxide from incineration.

In the present invention, reutilization of industrial waste, such as chicken feathers waste, into keratin, and fabrication into proton conductive membranes of possible use in fuel cells, protonic transistors, and water-splitting devices is demonstrated. Chicken feathers may be used as a starting material for fabricating proton conductive biomaterials. A protein-based membrane derived from industrial waste fabricated using environmentally green methods and imparted with proton conductive properties may be obtained and may be used in a hydrogen polymer electrolyte membrane fuel cell.

Keratin proteins may be extracted from a keratin source (such as industrial chicken feathers) using an environmentally friendly and scalable process and converted into amyloid fibrils that are subsequently processed into free-standing amyloid fibril membranes.

Naturally occurring bio-based materials, such as feather waste, which otherwise would be incinerated due to the lack of applications, serves as a promising material due to its unique chemistry. The methods of the present invention may be low cost, do not require harsh reagents, and valorises low value feather waste towards the essential goal of a clean and green future. This invention aims to reduce the overall cost of PEMFCs by lowering the cost of membranes, as well as their environmental footprint. Furthermore, in the present invention, the cost of producing proton conductive materials is kept low with the usage of industrial low value waste materials and an economical fabrication process. Compared to Nafion, the cost of production of keratin amyloid fibril protein membranes of the present invention may be significantly lower with the added benefit of utilizing non-hazardous chemical reagents.

Amyloid fibrils may be obtained from industrial chicken feathers which, by reusing industrial feather waste of low value which would otherwise be incinerated, would fulfil a circular economy of zero waste. In addition, the major advantage of using biomaterials is the possibility of aqueous processing which promotes environmental friendliness as compared to petroleum-derived polymers which usually requires the usage of organic solvents. Also, proton conduction is imparted through the modification of thiol into sulfonic acid groups with lower pKa than carboxylic acids, boasting higher conductivity at lower cost compared to the aforementioned materials, additionally demonstrating the powering of devices when applied to a fuel cell.

### Method of Obtaining Keratin Protein Isolate

The present invention relates to a method of obtaining keratin protein isolate from a keratin source, the method comprising:
(a) preparing a first mixture comprising keratin source, chaotropic solvent, and a first reducing agent;
(b) precipitating keratin protein from the first mixture;
(c) preparing a second mixture comprising the keratin protein and a second reducing agent; and
(d) precipitating keratin protein isolate from the second mixture.

In step (a) of the method, a first mixture comprising keratin source, chaotropic solvent, and a first reducing agent may be prepared.

The keratin source may be selected from the group consisting of hair, nails, feathers, claws, beaks, horns, hoofs, hooves, and wools. The keratin source may be from humans, birds and/or animals. The keratin source may be selected from the group consisting of hair, nails, feathers, claws, beaks, horns, hoofs, hooves, and wools from human, birds and/or animals. For example, the birds may be poultry, chickens, ducks, turkeys, pigeons, or any birds. For example, the animals may be selected from the group consisting of mammals, sheep, rabbit alpaca, llama, goats, horses, zebra, cow, deer, moose, reindeer, rhinoceros, buffalo, reptiles, tortoise, turtles, terrapin. The keratin source may be chicken feathers. The keratin source may be selected from human, birds and/or animals that may contain keratin or keratin-like proteins.

The chaotropic solvent may be selected from the group consisting of urea, thiourea, guanidinium chloride, lithium perchlorate, lithium acetate, and sodium dodecyl sulfate and combinations thereof.

The first reducing agent may be selected from the group consisting of ammonium thioglycolate, thiols, dithiothreitol, β-mercaptoethanol, 2-mercaptoethanol, dithiothreitol, 3-mercapto-1,2-propandiol, thioglycolic acid, tris(2-carboxyethyl)phosphine, tris(hydroxypropyl)phosphine, tris(hydroxymethyl)phosphine, and combinations thereof. In keratin, the cysteine of one keratin molecule forms a disulfide bond with the cysteine of a neighboring keratin molecule. However, with the addition of a reducing agent which contains thiol (—SH) groups, one of the sulfur atoms in the disulfide bond of keratin is replaced and hence the disulfide bonds in keratin are broken.

Step (a) of the method may comprise heating the first mixture at a temperature of about 40° C. to about 90° C., or about 50° C. to about 80° C. Step (a) of the method may be performed at a temperature in a range of about 40° C. to about 90° C., about 41° C. to about 90° C., about 42° C. to about 90° C., about 43° C. to about 90° C., about 44° C. to about 90° C., about 45° C. to about 90° C., about 46° C. to about 90° C., about 47° C. to about 90° C., about 48° C. to about 90° C., about 49° C. to about 90° C., about 50° C. to about 90° C., about 51° C. to about 90° C., about 52° C. to about 90° C., about 53° C. to about 90° C., about 54° C. to about 90° C., about 55° C. to about 90° C. about 56° C. to about 90° C., about 57° C. to about 90° C., about 58° C. to about 90° C., about 59° C. to about 90° C., about 60° C. to about 90° C., about 61° C. to about 90° C., about 62° C. to about 90° C., about 63° C. to about 90° C., about 64° C. to about 90° C., about 65° C. to about 90° C., about 66° C. to about 90° C., about 67° C. to about 90° C., about 68° C. to about 90° C., about 69° C. to about 90° C., about 70° C. to about 90° C., about 71° C. to about 90° C., about 72° C. to about 90° C., about 73° C. to about 90° C., about 74° C. to about 90° C., about 75° C. to about 90° C., about 76° C. to about 90° C., about 77° C. to about 90° C., about 78° C. to about 90° C., about 79° C. to about 90° C., about 80° C. to about 90° C., about 81° C. to about 90° C., about 82° C. to about 90° C., about 83° C. to about 90° C., about 84° C. to about 90° C., about 85° C. to about 90° C., about 86° C. to about 90° C., about 87° C. to about 90° C., about 88° C. to about 90° C., about 89° C. to about 90° C., about 40° C. to about 90° C., about 40° C. to about 89° C., about 40° C. to about 88° C., about 40° C. to about 87° C., about 40° C. to about 86° C., about 40° C. to about 85° C., about 40° C. to about 84° C., about 40° C. to about 83° C., about 40° C. to about 82° C., about 40° C. to about 81° C., about 40° C. to about 80° C., about 40° C. to about 79° C., about 40° C. to about 78° C., about 40° C. to about 77° C., about 40° C. to about 76° C., about 40° C. to about 75° C., about 40° C. to about 74° C., about 40° C. to about 73° C., about 40° C. to about 72° C., about 40° C. to about 71° C., about 40° C. to about 70° C., about 40° C. to about 69° C., about 40° C. to about 68° C., about 40° C. to about 67° C., about 40° C. to about 66° C., about 40° C. to about 65° C., about 40° C. to about 64° C., about 40° C. to about 63° C., about 40° C. to about 62° C., about 40° C. to about 61° C., about 40° C. to about 60° C., about 40° C. to about 59° C., about 40° C. to about 58° C., about 40° C. to about 57° C., about 40° C. to about 56° C., about 40° C. to about 55° C., about 40° C. to about 54° C., about 40° C. to about 53° C., about 40° C. to about 52° C., about 40° C. to about 51° C., about 40° C. to about 50° C., about 40° C. to about 49° C., about 40° C. to about 48° C., about 40° C. to about 47° C., about 40° C. to about 46° C., about 40° C. to about 45° C., about 40° C. to about 44° C., about 40° C. to about 43° C., about 40° C. to about 42° C., about 40° C. to about 41° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C. about 84° C., about 85° C., about 86° C., about 87° C., about 88° C., about 89° C., about 90° C., or any ranges or values therebetween.

Step (b) of the method comprises precipitating keratin protein from the first mixture. To precipitate the keratin protein, the pH of the first mixture may be adjusted and a first solution may be added. Step (b) of the method may comprise adjusting the pH of the first mixture to about pH 3 to about pH 5 and adding a first solution to precipitate the keratin protein.

The pH of the first mixture may be adjusted to be in the range of about 3 to about 5, about 3 to about 4.5, about 3 to about 4, about 3 to about 3.5, about 3.5 to about 5, about 3.5 to about 4.5, about 3.5 to about 4, about 4 to about 5, about 4 to about 4.5, about 4.5 to about 5, or at most about 3, at most about 3.5, at most about 4, or at most about 4.5, or at most about 5, or about 3, about 3.5, about 4, about 4.5, about 5, or any ranges or values therebetween.

The pH of the first mixture and/or the second mixture may be adjusted to a desired pH by
(a) first measuring a pH of the first/second mixture;
(b) adding an acid to the mixture to reduce the pH, or adding a base to the mixture to increase the pH; and
(c) measuring the pH of the first/second mixture and repeating step (b) to reach the desired pH.

The pH of the first mixture may be adjusted by any acid, for example hydrochloric acid, sulphuric/sulfuric acid, acetic acid, nitric acid, phosphoric acid and other similar acids.

The first solution may comprise a salt or base selected from the group consisting of ammonium sulfate, sodium hydroxide, potassium hydroxide, ammonium hydroxide, tris(hydroxymethyl)aminomethane, and combinations thereof.

After adjusting the pH, the first mixture may be added into the first solution to precipitate the keratin protein. The first solution may be cold and at a temperature in the range of about 4° C. to about 20° C. The first solution may be at a temperature in a range of about 4° C. to about 20° C., about 4° C. to about 19.9° C., about 4° C. to about 19.8° C., about 4° C. to about 19.7° C., about 4° C. to about 19.6° C., about 4° C. to about 19.5° C., about 4° C. to about 19.4° C., about 4° C. to about 19.3° C., about 4° C. to about 19.2° C., about 4° C. to about 19.1° C., about 4° C. to about 19° C., about 4° C. to about 18.9° C., about 4° C. to about 18.8° C., about 4° C. to about 18.7° C., about 4° C. to about 18.6° C., about 4° C. to about 18.5° C., about 4° C. to about 18.4° C., about 4° C. to about 18.3° C., about 4° C. to about 18.2° C., about 4° C. to about 18.1° C., about 4° C. to about 18° C., about 4° C. to about 17.9° C., about 4° C. to about 17.8° C., about 4° C. to about 17.7° C., about 4° C. to about 17.6° C., about 4° C. to about 17.5° C., about 4° C. to about 17.4° C., about 4° C. to about 17.3° C., about 4° C. to about 17.2° C., about 4° C. to about 17.1° C., about 4° C. to about 17° C., about 4° C. to about 16.9° C., about 4° C. to about 16.8° C., about 4° C. to about 16.7° C., about 4° C. to about 16.6° C., about 4° C. to about 16.5° C., about 4° C. to about 16.4° C., about 4° C. to about 16.3° C., about 4° C. to about 16.2° C., about 4° C. to about 16.1° C., about 4° C. to about 16° C., about 4° C. to about 15.9° C., about 4° C. to about 15.8° C., about 4° C. to about 15.7° C., about 4° C. to about 15.6° C., about 4° C. to about 15.5° C., about 4° C. to about 15.4° C., about 4° C. to about 15.3° C., about 4° C. to about 15.2° C., about 4° C. to about 15.1° C., about 4° C. to about 15° C., about 4° C. to about 14.9° C., about 4° C. to about 14.8° C., about 4° C. to about 14.7° C., about 4° C. to about 14.6° C., about 4° C. to about 14.5° C., about 4° C. to about 14.4° C., about 4° C. to about 14.3° C., about 4° C. to about 14.2° C., about 4° C. to about 14.1° C., about 4° C. to about 14° C., about 4° C. to about 13.9° C., about 4° C. to about 13.8° C., about 4° C. to about 13.7° C., about 4° C. to about 13.6° C., about 4° C. to about 13.5° C., about 4° C. to about 13.4° C., about 4° C. to about 13.3° C., about 4° C. to about 13.2° C., about 4° C. to about 13.1° C., about 4° C. to about 13° C., about 4° C. to about 12.9° C., about 4° C. to about 12.8° C., about 4° C. to about 12.7° C., about 4° C. to about 12.6° C., about 4° C. to about 12.5° C., about 4° C. to about 12.4° C., about 4° C. to about 12.3° C., about 4° C. to about 12.2° C., about 4° C. to about 12.1° C., about 4° C. to about 12° C., about 4° C. to about 11.9° C., about 4° C. to about 11.8° C., about 4° C. to about 11.7° C., about 4° C. to about 11.6° C., about 4° C. to about 11.5° C., about 4° C. to about 11.4° C., about 4° C. to about 11.3° C., about 4° C. to about 11.2° C., about 4° C. to about 11.1° C., about 4° C. to about 11° C., about 4° C. to about 10.9° C., about 4° C. to about 10.8° C., about 4° C. to about 10.7° C., about 4° C. to about 10.6° C., about 4° C. to about 10.5° C., about 4° C. to about 10.4° C., about 4° C. to about 10.3° C., about 4° C. to about 10.2° C., about 4° C. to about 10.1° C., about 4° C. to about 10° C., about 4° C. to about 9.9° C. about 4° C. to about 9.8° C., about 4° C. to about 9.7° C., about 4° C. to about 9.6° C., about 4° C. to about 9.5° C., about 4° C. to about 9.4° C., about 4° C. to about 9.3° C., about 4° C. to about 9.2° C., about 4° C. to about 9.1° C., about 4° C. to about 9° C., about 4° C. to about 8.9° C. about 4° C. to about 8.8° C., about 4° C. to about 8.7° C., about 4° C. to about 8.6° C., about 4° C. to about 8.5° C., about 4° C. to about 8.4° C., about 4° C. to about 8.3° C., about 4° C. to about 8.2° C., about 4° C. to about 8.1° C., about 4° C. to about 8° C., about 4° C. to about 7.9° C., about 4° C. to about 7.8° C. about 4° C. to about 7.7° C., about 4° C. to about 7.6° C., about 4° C. to about 7.5° C., about 4° C. to about 7.4° C., about 4° C. to about 7.3° C., about 4° C. to about 7.2° C., about 4° C. to about 7.1° C., about 4° C. to about 7° C., about 4° C. to about 6.9° C., about 4° C. to about 6.8° C., about 4° C. to about 6.7° C., about 4° C. to about 6.6° C., about 4° C. to about 6.5° C., about 4° C. to about 6.4° C., about 4° C. to about 6.3° C., about 4° C. to about 6.2° C., about 4° C. to about 6.1° C., about 4° C. to about 6° C., about 4° C. to about 5.9° C., about 4° C. to about 5.8° C., about 4° C. to about 5.7° C., about 4° C. to about 5.6° C., about 4° C. to about 5.5° C., about 4° C. to about 5.4° C., about 4° C. to about 5.3° C., about 4° C. to about 5.2° C., about 4° C. to about 5.1° C., about 4° C. to about 5° C., about 4° C. to about 4.9° C., about 4° C. to about 4.8° C., about 4° C. to about 4.7° C., about 4° C. to about 4.6° C., about 4° C. to about 4.5° C., about 4° C. to about 4.4° C., about 4° C. to about 4.3° C., about 4° C. to about 4.2° C., about 4° C. to about 4.1° C., about 4° C. to about 20° C., about 4.1° C. to about 20° C., about 4.2° C. to about 20° C., about 4.3° C. to about 20° C. about 4.4° C. to about 20° C., about 4.5° C. to about 20° C. about 4.6° C. to about 20° C., about 4.7° C. to about 20° C., about 4.8° C. to about 20° C., about 4.9° C. to about 20° C., about 5° C. to about 20° C., about 5.1° C. to about 20° C., about 5.2° C. to about 20° C., about 5.3° C. to about 20° C. about 5.4° C. to about 20° C., about 5.5° C. to about 20° C. about 5.6° C. to about 20° C., about 5.7° C. to about 20° C., about 5.8° C. to about 20° C., about 5.9° C. to about 20° C., about 6° C. to about 20° C., about 6.1° C. to about 20° C., about 6.2° C. to about 20° C., about 6.3° C. to about 20° C., about 6.4° C. to about 20° C., about 6.5° C. to about 20° C., about 6.6° C. to about 20° C., about 6.7° C. to about 20° C., about 6.8° C. to about 20° C., about 6.9° C. to about 20° C., about 7° C. to about 20° C., about 7.1° C. to about 20° C., about 7.2° C. to about 20° C., about 7.3° C. to about 20° C., about 7.4° C. to about 20° C., about 7.5° C. to about 20° C., about 7.6° C. to about 20° C., about 7.7° C. to about 20° C., about 7.8° C. to about 20° C., about 7.9° C. to about 20° C., about 8° C. to about 20° C., about 8.1° C. to about 20° C., about 8.2° C. to about 20° C., about 8.3° C. to about 20° C., about 8.4° C. to about 20° C., about 8.5° C. to about 20° C., about 8.6° C. to about 20° C., about 8.7° C. to about 20° C., about 8.8° C. to about 20° C., about 8.9° C. to about 20° C., about 9° C. to about 20° C., about 9.1° C. to about 20° C., about 9.2° C. to about 20° C., about 9.3° C. to about 20° C., about 9.4° C. to about 20° C., about 9.5° C. to about 20° C., about 9.6° C. to about 20° C., about 9.7° C. to about 20° C., about 9.8° C. to about 20° C., about 9.9° C. to about 20° C. about 10° C. to about 20° C., about 10.1° C. to about 20° C., about 10.2° C. to about 20° C., about 10.3° C. to about 20° C., about 10.4° C. to about 20° C., about 10.5° C. to about 20° C., about 10.6° C. to about 20° C., about 10.7° C. to about 20° C., about 10.8° C. to about 20° C., about 10.9° C. to about 20° C., about 11° C. to about 20° C., about 11.1° C. to about 20° C., about 11.2° C. to about 20° C., about 11.3° C. to about 20° C., about 11.4° C. to about 20° C., about 11.5° C. to about 20° C., about 11.6° C. to about 20° C., about 11.7° C. to about 20° C., about 11.8° C. to about 20° C., about 11.9° C. to about 20° C., about 12° C. to about 20° C., about 12.1° C. to about 20° C., about 12.2° C. to about 20° C., about 12.3° C. to about 20° C., about 12.4° C. to about 20° C., about 12.5° C. to about 20° C., about 12.6° C. to about 20° C., about 12.7° C. to about 20° C., about 12.8° C. to about 20° C., about 12.9° C. to about 20° C., about 13° C. to about 20° C., about 13.1° C. to about 20° C., about 13.2° C. to about 20° C., about 13.3° C. to about 20° C., about 13.4° C. to about 20° C., about 13.5° C. to about 20° C. about 13.6° C. to about 20° C., about 13.7° C. to about 20° C., about 13.8° C. to about 20° C., about 13.9° C. to about 20° C., about 14° C. to about 20° C., about 14.1° C. to about 20° C., about 14.2° C. to about 20° C., about 14.3° C. to about 20° C., about 14.4° C. to about 20° C., about 14.5° C. to about 20° C., about 14.6° C. to about 20° C. about 14.7° C. to about 20° C., about 14.8° C. to about 20° C., about 14.9° C. to about 20° C., about 15° C. to about 20° C., about 15.1° C. to about 20° C. about 15.2° C. to about 20° C. about 15.3° C. to about 20° C. about 15.4° C. to about 20° C. about 15.5° C. to about 20° C., about 15.6° C. to about 20° C., about 15.7° C. to about 20° C., about 15.8° C. to about 20° C., about 15.9° C. to about 20° C., about 16° C. to about 20° C., about 16.1° C. to about 20° C., about 16.2° C. to about 20° C., about 16.3° C. to about 20° C., about 16.4° C. to about 20° C., about 16.5° C. to about 20° C., about 16.6° C. to about 20° C., about 16.7° C. to about 20° C., about 16.8° C. to about 20° C., about 16.9° C. to about 20° C., about 17° C. to about 20° C., about 17.1° C. to about 20° C., about 17.2° C. to about 20° C., about 17.3° C. to about 20° C., about 17.4° C. to about 20° C., about 17.5° C. to about 20° C., about 17.6° C. to about 20° C., about 17.7° C. to about 20° C., about 17.8° C. to about 20° C., about 17.9° C. to about 20° C., about 18° C. to about 20° C., about 18.1° C. to about 20° C., about 18.2° C. to about 20° C., about 18.3° C. to about 20° C., about 18.4° C. to about 20° C., about 18.5° C. to about 20° C., about 18.6° C. to about 20° C., about 18.7° C. to about 20° C., about 18.8° C. to about 20° C., about 18.9° C. to about 20° C., about 19° C. to about 20° C., about 19.1° C. to about 20° C., about 19.2° C. to about 20° C., about 19.3° C. to about 20° C., about 19.4° C. to about 20° C., about 19.5° C. to about 20° C., about 19.6° C. to about 20° C., about 19.7° C. to about 20° C., about 19.8° C. to about 20° C., about 19.9° C. to about 20° C., about 4° C. about 4.1° C., about 4.2° C., about 4.3° C., about 4.4° C., about 4.5° C., about 4.6° C., about 4.7° C., about 4.8° C., about 4.9° C., about 5° C., about 5.1° C., about 5.2° C., about 5.3° C., about 5.4° C., about 5.5° C., about 5.6° C., about 5.7° C., about 5.8° C., about 5.9° C., about 6° C. about 6.1° C., about 6.2° C., about 6.3° C., about 6.4° C., about 6.5° C., about 6.6° C., about 6.7° C., about 6.8° C., about 6.9° C., about 7° C., about 7.1° C., about 7.2° C., about 7.3° C., about 7.4° C., about 7.5° C., about 7.6° C., about 7.7° C., about 7.8° C., about 7.9° C., about 8° C., about 8.1° C., about 8.2° C., about 8.3° C., about 8.4° C., about 8.5° C., about 8.6° C., about 8.7° C., about 8.8° C., about 8.9° C., about 9° C., about 9.1° C., about 9.2° C., about 9.3° C., about 9.4° C., about 9.5° C., about 9.6° C., about 9.7° C., about 9.8° C., about 9.9° C., about 10° C., about 10.1° C., about 10.2° C., about 10.3° C., about 10.4° C., about 10.5° C., about 10.6° C., about 10.7° C., about 10.8° C., about 10.9° C., about 11° C., about 11.1° C., about 11.2° C., about 11.3° C., about 11.4° C., about 11.5° C., about 11.6° C., about 11.7° C., about 11.8° C., about 11.9° C., about 12° C., about 12.1° C., about 12.2° C., about 12.3° C., about 12.4° C., about 12.5° C., about 12.6° C., about 12.7° C., about 12.8° C., about 12.9° C., about 13° C., about 13.1° C., about 13.2° C., about 13.3° C., about 13.4° C., about 13.5° C., about 13.6° C., about 13.7° C., about 13.8° C., about 13.9° C., about 14° C., about 14.1° C., about 14.2° C., about 14.3° C., about 14.4° C., about 14.5° C., about 14.6° C., about 14.7° C. about 14.8° C. about 14.9° C. about 15° C., about 15.1° C., about 15.2° C., about 15.3° C., about 15.4° C., about 15.5° C., about 15.6° C., about 15.7° C., about 15.8° C., about 15.9° C., about 16° C., about 16.1° C., about 16.2° C., about 16.3° C., about 16.4° C., about 16.5° C., about 16.6° C., about 16.7° C., about 16.8° C., about 16.9° C., about 17° C., about 17.1° C., about 17.2° C., about 17.3° C., about 17.4° C., about 17.5° C., about 17.6° C., about 17.7° C., about 17.8° C., about 17.9° C., about 18° C., about 18.1° C., about 18.2° C., about 18.3° C., about 18.4° C., about 18.5° C., about 18.6° C., about 18.7° C., about 18.8° C., about 18.9° C., about 19° C., about 19.1° C., about 19.2° C., about 19.3° C., about 19.4° C., about 19.5° C., about 19.6° C., about 19.7° C., about 19.8° C., about 19.9° C., about 20° C., or any ranges or values therebetween.

The volume of the first solution may be at a volume in the range of about 5 times to about 10 times of the first mixture. The volume of the first solution may be at a volume in the range of about 5 times to about 10 times of the first mixture, about 5 times to about 9.9 times, about 5 times to about 9.8 times, about 5 times to about 9.7 times, about 5 times to about 9.6 times, about 5 times to about 9.5 times, about 5 times to about 9.4 times, about 5 times to about 9.3 times, about 5 times to about 9.2 times, about 5 times to about 9.1 times, about 5 times to about 9 times, about 5 times to about 8.9 times, about 5 times to about 8.8 times, about 5 times to about 8.7 times, about 5 times to about 8.6 times, about 5 times to about 8.5 times, about 5 times to about 8.4 times, about 5 times to about 8.3 times, about 5 times to about 8.2 times, about 5 times to about 8.1 times, about 5 times to about 8 times, about 5 times to about 7.9 times, about 5 times to about 7.8 times, about 5 times to about 7.7 times, about 5 times to about 7.6 times, about 5 times to about 7.5 times, about 5 times to about 7.4 times, about 5 times to about 7.3 times, about 5 times to about 7.2 times, about 5 times to about 7.1 times, about 5 times to about 7 times, about 5 times to about 6.9 times, about 5 times to about 6.8 times, about 5 times to about 6.7 times, about 5 times to about 6.6 times, about 5 times to about 6.5 times, about 5 times to about 6.4 times, about 5 times to about 6.3 times, about 5 times to about 6.2 times, about 5 times to about 6.1 times, about 5 times to about 6 times, about 5 times to about 5.9 times, about 5 times to about 5.8 times, about 5 times to about 5.7 times, about 5 times to about 5.6 times, about 5 times to about 5.5 times, about 5 times to about 5.4 times, about 5 times to about 5.3 times, about 5 times to about 5.2 times, about 5 times to about 5.1 times, about 5 times to about 10 times, about 5.1 times to about 10 times, about 5.2 times to about 10 times, about 5.3 times to about 10 times, about 5.4 times to about 10 times, about 5.5 times to about 10 times, about 5.6 times to about 10 times, about 5.7 times to about 10 times, about 5.8 times to about 10 times, about 5.9 times to about 10 times, about 6 times to about 10 times, about 6.1 times to about 10 times, about 6.2 times to about 10 times, about 6.3 times to about 10 times, about 6.4 times to about 10 times, about 6.5 times to about 10 times, about 6.6 times to about 10 times, about 6.7 times to about 10 times, about 6.8 times to about 10 times, about 6.9 times to about 10 times, about 7 times to about 10 times, about 7.1 times to about 10 times, about 7.2 times to about 10 times, about 7.3 times to about 10 times, about 7.4 times to about 10 times, about 7.5 times to about 10 times, about 7.6 times to about 10 times, about 7.7 times to about 10 times, about 7.8 times to about 10 times, about 7.9 times to about 10 times, about 8 times to about 10 times, about 8.1 times to about 10 times, about 8.2 times to about 10 times, about 8.3 times to about 10 times, about 8.4 times to about 10 times, about 8.5 times to about 10 times, about 8.6 times to about 10 times, about 8.7 times to about 10 times, about 8.8 times to about 10 times, about 8.9 times to about 10 times, about 9 times to about 10 times, about 9.1 times to about 10 times, about 9.2 times to about 10 times, about 9.3 times to about 10 times, about 9.4 times to about 10 times, about 9.5 times to about 10 times, about 9.6 times to about 10 times, about 9.7 times to about 10 times, about 9.8 times to about 10 times, about 9.9 times to about 10 times, about 5 times, about 5.1 times, about 5.2 times, about 5.3 times, about 5.4 times, about 5.5 times, about 5.6 times, about 5.7 times, about 5.8 times, about 5.9 times, about 6 times, about 6.1 times, about 6.2 times, about 6.3 times, about 6.4 times, about 6.5 times, about 6.6 times, about 6.7 times, about 6.8 times, about 6.9 times, about 7 times, about 7.1 times, about 7.2 times, about 7.3 times, about 7.4 times, about 7.5 times, about 7.6 times, about 7.7 times, about 7.8 times, about 7.9 times, about 8 times, about 8.1 times, about 8.2 times, about 8.3 times, about 8.4 times, about 8.5 times, about 8.6 times, about 8.7 times, about 8.8 times, about 8.9 times, about 9 times, about 9.1 times, about 9.2 times, about 9.3 times, about 9.4 times, about 9.5 times, about 9.6 times, about 9.7 times, about 9.8 times, about 9.9 times, about 10 times, of the first mixture, or any ranges or values therebetween.

The method may further comprise freeze drying the keratin protein after step (b). Freeze drying of keratin proteins after step (b) may remove water and/or other solvents or compounds from the keratin protein. Freeze drying of keratin proteins also allows the accurate weighing of the keratin protein for an appropriate concentration of the keratin protein for step (c).

In step (c), a second mixture comprising the keratin protein and a second reducing agent may be prepared. The second reducing agent may be selected from the group consisting of ammonium thioglycolate, thiols, dithiothreitol, β-mercaptoethanol, 2-mercaptoethanol, dithiothreitol, 3-mercapto-1,2-propandiol, thioglycolic acid, tris(2-carboxyethyl)phosphine, tris(hydroxypropyl)phosphine, tris(hydroxymethyl)phosphine, and combinations thereof. The first and second reducing agent may be the same or different. The first and second reducing agent may be independently selected from the group consisting of ammonium thioglycolate, thiols, dithiothreitol, β-mercaptoethanol, 2-mercaptoethanol, dithiothreitol, 3-mercapto-1,2-propandiol, thioglycolic acid, tris(2-carboxyethyl)phosphine, tris(hydroxypropyl)phosphine, tris(hydroxymethyl)phosphine, and combinations thereof.

Step (c) of the method may further comprise dissolving the keratin protein in a second solution and subsequently adding the second reducing agent.

The second solution may comprise a salt or base selected from the group consisting of ammonium sulfate, sodium hydroxide, potassium hydroxide, ammonium hydroxide, and tris(hydroxymethyl)aminomethane and combinations thereof. The first and second solutions may be the same or may be different. The first and second solutions may comprise a salt or base independently selected from the group consisting of ammonium sulfate, sodium hydroxide, potassium hydroxide, ammonium hydroxide, tris(hydroxymethyl)aminomethane and combinations thereof.

Step (d) may comprise precipitating keratin protein isolate from the second mixture. To precipitate the keratin protein isolate, the pH of the second mixture may be adjusted. Step (d) of the method may comprise adjusting the pH of the second mixture to about pH 3 to about pH 5 to precipitate the keratin protein isolate. The pH of the second mixture may be adjusted to be in the range of about 3 to about 5, about 3 to about 4.5, about 3 to about 4, about 3 to about 3.5, about 3.5 to about 5, about 3.5 to about 4.5, about 3.5 to about 4, about 4 to about 5, about 4 to about 4.5, about 4.5 to about 5, or at most about 3, at most about 3.5, at most about 4, or at most about 4.5, or at most about 5, or about 3, about 3.5, about 4, about 4.5, about 5, or any ranges or values therebetween.

The method may further comprise freeze drying the keratin protein isolate after step (d). Freeze drying of keratin proteins after step (d) may remove water from the proteins and may allow the freeze-dried proteins to have long-term stability and shelf-life, which may offer advantages for storage as well as for shipping and distribution. The freeze-dried proteins may have longer stability at ambient temperatures.

The first solution and second solution may comprise a base or a salt. The first solution may be a first basic solution; and the second solution may be a second basic solution. The first and second solution may be independently selected from the group consisting of ammonium sulfate, sodium hydroxide, potassium hydroxide, ammonium hydroxide, and tris(hydroxymethyl)aminomethane and combinations thereof.

### Method of Obtaining Keratin Amyloid Fibrils

The present invention also relates to a method of obtaining keratin amyloid fibrils, the method comprising:
(i) obtaining keratin protein isolate as disclosed above;
(ii) preparing a third mixture comprising keratin protein isolate, acid solution, and a third reducing agent; and
(iii) subjecting the third mixture to heat treatment to obtain keratin amyloid fibrils.

Keratin protein isolate may be obtained as disclosed above and may be used for preparing a third mixture. The third mixture comprising keratin protein isolate, acid solution and a third reducing agent may be prepared.

The acid solution may be selected from the group consisting of acetic acid, formic acid, propionic acid, tartaric acid, malonic acid, oxalic acid, pyruvic acid, and citric acid and combinations thereof.

The third reducing agent may be selected from the group consisting of ammonium thioglycolate, thiols, dithiothreitol, β-mercaptoethanol, 2-mercaptoethanol, dithiothreitol, 3-mercapto-1,2-propandiol, thioglycolic acid, tris(2-carboxyethyl)phosphine, tris(hydroxypropyl)phosphine, and tris(hydroxymethyl)phosphine and combinations thereof. The third reducing agent may be the same or different with the first and/or second reducing agent. The first, second and third reducing agent may be independently selected from the group consisting of ammonium thioglycolate, thiols, dithiothreitol, s-mercaptoethanol, 2-mercaptoethanol, dithiothreitol, 3-mercapto-1,2-propandiol, thioglycolic acid, tris(2-carboxyethyl)phosphine, tris(hydroxypropyl)phosphine, and tris(hydroxymethyl)phosphine and combinations thereof.

In step (iii) of the method, the third mixture may be subjected to heat treatment to obtain keratin amyloid fibrils.

Step (iii) may comprise heating the third mixture at a temperature of about 80° C. to about 100° C. Step (iii) of the method may comprise heating the third mixture at a temperature of about 80° C. to about 100° C. Step (iii) of the method may be performed at a temperature in a range of about 80° C. to about 100° C., about 81° C. to about 100° C., about 82° C. to about 100° C., about 83° C. to about 100° C., about 84° C. to about 100° C. about 85° C. to about 100° C., about 86° C. to about 100° C., about 87° C. to about 100° C., about 88° C. to about 100° C., about 89° C. to about 100° C., about 90° C. to about 100° C., about 91° C. to about 100° C., about 92° C. to about 100° C., about 93° C. to about 100° C., about 94° C. to about 100° C., about 95° C. to about 100° C., about 96° C. to about 100° C., about 97° C. to about 100° C., about 98° C. to about 100° C., about 99° C. to about 100° C., about 80° C. to about 100° C., about 80° C. to about 99° C., about 80° C. to about 98° C., about 80° C. to about 97° C., about 80° C. to about 96° C., about 80° C. to about 95° C., about 80° C. to about 94° C., about 80° C. to about 93° C., about 80° C. to about 92° C., about 80° C. to about 91° C., about 80° C. to about 90° C., about 80° C. to about 89° C., about 80° C. to about 88° C., about 80° C. to about 87° C., about 80° C. to about 86° C., about 80° C. to about 85° C., about 80° C. to about 84° C., about 80° C. to about 83° C., about 80° C. to about 82° C., about 80° C. to about 81° C., about 80° C., about 81° C., about 82° C., about 83° C., about 84° C., about 85° C., about 86° C., about 87° C. about 88° C., about 89° C. about 90° C. about 91° C. about 92° C. about 93° C. about 94° C., about 95° C., about 96° C., about 97° C., about 98° C., about 99° C., about 100° C., or any ranges or values therebetween.

Step (iii) may be performed for about 2 hours to about 24 hours. The heat treatment may be performed for a duration in the range of about 2 hours to about 24 hours, about 2 hours to about 23.5 hours, about 2 hours to about 23 hours, about 2 hours to about 22.5 hours, about 2 hours to about 22 hours, about 2 hours to about 21 hours, about 2 hours to about 20.5 hours, about 2 hours to about 20 hours, about 2 hours to about 19.5 hours, about 2 hours to about 19 hours, about 2 hours to about 18.5 hours, about 2 hours to about 18 hours, about 2 hours to about 17.5 hours, about 2 hours to about 17 hours, about 2 hours to about 16.5 hours, about 2 hours to about 16 hours, about 2 hours to about 15.5 hours, about 2 hours to about 15 hours, about 2 hours to about 14.5 hours, about 2 hours to about 14 hours, about 2 hours to about 13.5 hours, about 2 hours to about 13 hours, about 2 hours to about 12.5 hours, about 2 hours to about 12 hours, about 2 hours to about 11.5 hours, about 2 hours to about 11 hours, about 2 hours to about 10.5 hours, about 2 hours to about 10 hours, about 2 hours to about 9.5 hours, about 2 hours to about 9 hours, about 2 hours to about 8.5 hours, about 2 hours to about 8 hours, about 2 hours to about 7.5 hours, about 2 hours to about 7 hours, about 2 hours to about 6.5 hours, about 2 hours to about 6 hours, about 2 hours to about 5.5 hours, about 2 hours to about 5 hours, about 2 hours to about 4.5 hours, about 2 hours to about 4 hours, about 2 hours to about 3.5 hours, about 2 hours to about 3 hours, about 2 hours to about 2.5 hours, about 2 hours to about 24 hours, about 2.5 hours to about 24 hours, about 3 hours to about 24 hours, about 3.5 hours to about 24 hours, about 4 hours to about 24 hours, about 4.5 hours to about 24 hours, about 5 hours to about 24 hours, about 5.5 hours to about 24 hours, about 6 hours to about 24 hours, about 6.5 hours to about 24 hours, about 7 hours to about 24 hours, about 7.5 hours to about 24 hours, about 8 hours to about 24 hours, about 8.5 hours to about 24 hours, about 9 hours to about 24 hours, about 9.5 hours to about 24 hours, about 10 hours to about 24 hours, about 10.5 hours to about 24 hours, about 11 hours to about 24 hours, about 11.5 hours to about 24 hours, about 12 hours to about 24 hours, about 12.5 hours to about 24 hours, about 13 hours to about 24 hours, about 13.5 hours to about 24 hours, about 14 hours to about 24 hours, about 14.5 hours to about 24 hours, about 15 hours to about 24 hours, about 15.5 hours to about 24 hours, about 16 hours to about 24 hours, about 16.5 hours to about 24 hours, about 17 hours to about 24 hours, about 17.5 hours to about 24 hours, about 18 hours to about 24 hours, about 18.5 hours to about 24 hours, about 19 hours to about 24 hours, about 19.5 hours to about 24 hours, about 20 hours to about 24 hours, about 20.5 hours to about 24 hours, about 21 hours to about 24 hours, about 21.5 hours to about 24 hours, about 22 hours to about 24 hours, about 22.5 hours to about 24 hours, about 23 hours to about 24 hours, about 23.5 hours to about 24 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, or any ranges or values therebetween.

### Method of Fabricating a Membrane

The keratin amyloid fibrils may then be further processed into membranes for electrochemical applications or other applications related to proton transport (e.g. proton conductive cell).

The present invention includes a method of fabricating a membrane for an electrochemical cell, the method comprising:
(A) forming a film by combining keratin amyloid fibrils as disclosed herein, a crosslinking agent, and a dopant;
(B) heat treating the film to induce crosslinking; and
(C) subjecting the crosslinked film to oxidation and protonation to form the membrane.

In step (A) of the method, a film may be formed by combining keratin amyloid fibrils, a crosslinking agent, and a dopant.

The film may be formed by combining keratin amyloid fibrils, a crosslinking agent, and a dopant. The film may be a “freestanding” film or a film applied on a substrate. After keratin amyloid fibrils, crosslinking agent and dopant are combined, they may be left to dry to form a film. The film may be peeled off the surface it is left to dry on to become a “freestanding” film.

In another embodiment, the film may be formed on a substrate by applying keratin amyloid fibrils, crosslinking agent and dopant on a substrate.

The present invention also relates to a method of fabricating a membrane for an electrochemical cell, the method comprising:
(A) forming a film on a substrate by applying keratin amyloid fibrils as disclosed herein, a crosslinking agent, and a dopant onto a substrate;
(B) heat treating the film to induce crosslinking; and
(C) subjecting the crosslinked film to oxidation and protonation to form the membrane.

In step (A) of the method, a film may be formed by applying keratin amyloid fibrils, a crosslinking agent, and a dopant onto a substrate. The substrate may be a hydrophobic surface or a plastic surface. The substrate may be of a smooth surface. The substrate may be a highly ordered pyrolytic graphite (HOPG) substrate. The substrate may also be a glass, a quartz, a mica substrate, a silicon wafer, an amorphous carbon layer, an indium tin oxide layer or alike.

The crosslinking agent may be selected from the group consisting of formaldehyde, glyoxal, malondialdehyde, succindialdehyde, and glutaraldehyde and combinations thereof.

The dopant may be selected from the group consisting of mercaptosuccinic acid, mercaptoacetic acid, and 3-mercaptopropionic acid and combinations thereof.

Step (B) comprises heat treating the film to induce crosslinking.

Step (B) may comprise heating the film at a temperature of about 150° C. to about 180° C. Step (B) may comprise heating the film at a temperature in a range of about 150° C. to about 180° C., about 151° C. to about 180° C., about 152° C. to about 180° C., about 153° C. to about 180° C, about 154° C. to about 180° C. about 155° C. to about 180° C. about 156° C. to about 180° C. about 157° C. to about 180° C., about 158° C. to about 180° C., about 159° C. to about 180° C., about 160° C. to about 180° C., about 161° C. to about 180° C., about 162° C. to about 180° C., about 163° C. to about 180° C. about 164° C. to about 180° C., about 165° C. to about 180° C., about 166° C. to about 180° C., about 167° C. to about 180° C., about 168° C. to about 180° C., about 169° C. to about 180° C., about 170° C. to about 180° C., about 171° C. to about 180° C., about 172° C. to about 180° C., about 173° C. to about 180° C., about 174° C. to about 180° C., about 175° C. to about 180° C., about 176° C. to about 180° C., about 177° C. to about 180° C., about 178° C. to about 180° C., about 179° C. to about 180° C., about 150° C. to about 180° C., about 150° C. to about 179° C., about 150° C. to about 178° C., about 150° C. to about 177° C., about 150° C. to about 176° C., about 150° C. to about 175° C., about 150° C. to about 174° C., about 150° C. to about 173° C., about 150° C. to about 172° C., about 150° C. to about 171° C., about 150° C. to about 170° C., about 150° C. to about 169° C., about 150° C. to about 168° C., about 150° C. to about 167° C., about 150° C. to about 166° C., about 150° C. to about 165° C., about 150° C. to about 164° C., about 150° C. to about 163° C., about 150° C. to about 162° C., about 150° C. to about 161° C., about 150° C. to about 160° C., about 150° C. to about 159° C., about 150° C. to about 158° C., about 150° C. to about 157° C., about 150° C. to about 156° C., about 150° C. to about 155° C., about 150° C. to about 154° C., about 150° C. to about 153° C., about 150° C. to about 152° C., about 150° C. to about 151° C., about 150° C., about 151° C., about 152° C., about 153° C., about 154° C., about 155° C., about 156° C., about 157° C., about 158° C., about 159° C., about 160° C., about 161° C., about 162° C., about 163° C., about 164° C., about 165° C., about 166° C., about 167° C., about 168° C., about 169° C., about 170° C., about 171° C., about 172° C., about 173° C., about 174° C., about 175° C., about 176° C., about 177° C., about 178° C., about 179° C., about 180° C. or any ranges or values therebetween.

Step (B) may be performed for about 30 minutes to about 60 minutes. Step (B) of the method may be performed for a duration in the range of about 30 minutes to about 60 minutes, about 31 minutes to about 60 minutes, about 32 minutes to about 60 minutes, about 33 minutes to about 60 minutes, about 34 minutes to about 60 minutes, about 35 minutes to about 60 minutes, about 36 minutes to about 60 minutes, about 37 minutes to about 60 minutes, about 38 minutes to about 60 minutes, about 39 minutes to about 60 minutes, about 40 minutes to about 60 minutes, about 41 minutes to about 60 minutes, about 42 minutes to about 60 minutes, about 43 minutes to about 60 minutes, about 44 minutes to about 60 minutes, about 45 minutes to about 60 minutes, about 46 minutes to about 60 minutes, about 47 minutes to about 60 minutes, about 48 minutes to about 60 minutes, about 49 minutes to about 60 minutes, about 50 minutes to about 60 minutes, about 51 minutes to about 60 minutes, about 52 minutes to about 60 minutes, about 53 minutes to about 60 minutes, about 54 minutes to about 60 minutes, about 55 minutes to about 60 minutes, about 56 minutes to about 60 minutes, about 57 minutes to about 60 minutes, about 58 minutes to about 60 minutes, about 59 minutes to about 60 minutes, about 30 minutes to about 59 minutes, about 30 minutes to about 58 minutes, about 30 minutes to about 57 minutes, about 30 minutes to about 56 minutes, about 30 minutes to about 55 minutes, about 30 minutes to about 54 minutes, about 30 minutes to about 53 minutes, about 30 minutes to about 52 minutes, about 30 minutes to about 51 minutes, about 30 minutes to about 50 minutes, about 30 minutes to about 49 minutes, about 30 minutes to about 48 minutes, about 30 minutes to about 47 minutes, about 30 minutes to about 46 minutes, about 30 minutes to about 45 minutes, about 30 minutes to about 44 minutes, about 30 minutes to about 43 minutes, about 30 minutes to about 42 minutes, about 30 minutes to about 41 minutes, about 30 minutes to about 40 minutes, about 30 minutes to about 39 minutes, about 30 minutes to about 38 minutes, about 30 minutes to about 37 minutes, about 30 minutes to about 36 minutes, about 30 minutes to about 35 minutes, about 30 minutes to about 34 minutes, about 30 minutes to about 33 minutes, about 30 minutes to about 32 minutes, about 30 minutes to about 31 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, or any ranges or values therebetween.

Step (C) may comprise subjecting the crosslinked film to oxidation and protonation to form the membrane.

For oxidation, step (C) of the method may comprise immersing the crosslinked film in an oxidizing agent to undergo oxidation. During oxidation, an —S terminal of a cysteine may be modified, where a thiol group of a cysteine compound may be converted into sulfonic acid groups through oxidation. The oxidizing agent may be selected from the group consisting of consisting of peroxide, peracctic acid, peroxy acids including performic acid, peracctic acid, peroxymonosulfuric acid, and peroxymonophosphoric acid.

For protonation, step (C) of the method may comprise immersing the crosslinked film in an acid. The crosslinked film comprises sulfonic acid groups. By adding an acid, the sulfonic acid groups are protonated which will allow the film to conduct protons.

The acid may be selected from the group consisting of organic acids, hydrochloric acid, sulfuric acid, sulfonic acids, phosphoric acid, nitric acid, to protonate the membrane.

Functional groups such as phosphoric acid, sulfonic acid, and carboxylic acid may inherit proton conductive properties through deprotonation with hydration. Among these groups, sulfonic acid groups possess the lowest pKa, which may ensure the highest degree of deprotonation and thus better transport of protons.

In step (A), the film may be formed by applying keratin amyloid fibrils as disclosed herein, a crosslinking agent, and a dopant onto a substrate. The components and formulation of either amyloid fibrils, crosslinking agent and/or dopant may be tuned to form a film and membrane of different properties. For example, the concentration of amyloid fibrils used can be varied to tune the thickness of the film and in turn the membrane. An increase in thickness would grant a sturdier membrane while also increasing the resistance and hence voltage drop in the fuel cell. Another example would be that the crosslinking concentration can be varied to increase the mechanical properties, while different additives containing thiol groups can also be varied or introduced to boost the proton conductive properties. Additional techniques to form the film may be employed such as solution casting, extrusion or electrospinning.

### Keratin Source to Membrane for Electrochemical Applications

Keratin may be obtained from chicken feathers via a fast and economical process (using low-cost industrial reagents without dialysis) and converted into amyloid fibrils upon heat treatment. Obtained fibrils may be further processed into membranes with imparted proton conductivity through a simple oxidative method. The amyloid fibril protein membrane derived from industrial feather waste may be further treated with imparted proton conductive properties and then applied in a fuel cell. Proton conductive properties can be imparted through a post oxidative treatment which convert Cys thiols into sulfonic acid groups, using a benign environmental process with harmless and inexpensive chemical compounds.

The performance of the membrane is demonstrated in a fuel cell device capable to transform hydrogen (H₂) and oxygen (O₂, directly from air) by electrochemical reaction into electrical power and mechanical work with water (H₂O) as sole by-product. To demonstrate the general potential of this approach, two additional applications of these materials, as protonic field-effect transistors, as well as in the generation of H₂ via water splitting, are discussed in the example section below.

Among low-cost biological raw materials, feather keratin protein serves as a promising candidate due to its high-volume production in the poultry industry, and in addition to its thiol-rich nature, allows the conversion of thiol into sulfonic acid groups through oxidation. This invention consists of the production of keratin amyloid fibril membranes starting from industrial feather waste, casting to produce a film, and converting the thiol groups into sulfonic acid groups through an oxidation process.

The product can be used in any application utilizing proton conductivity such as but not limited to fuel cells, electrolysers (i.e. water splitting), and transistors.

The functionality of the membranes is demonstrated by assembling them into a hydrogen fuel cell capable of powering several types of devices using hydrogen and air as fuel. Additionally, the same membranes could be used to generate hydrogen by water splitting, as well as in protonic field-effect transistors to modulate protonic conductivity via the electrostatic gating effect.

### EXAMPLES

Non-limiting examples of the invention will be further described in greater detail by reference to specific Examples, which should not be construed as in any way limiting the scope of the invention.

### Example 1: Materials and Methods

### (A) Protein Extraction and Isolation

Chicken feathers obtained from a local farm were washed and added to a keratin extraction solution (8 M urea containing 5 wt. % ammonium thioglycolate, pH adjusted to 9.8) at a ratio of 1:30 (w/v). The mixture was heated at 60° C. for 6 hours, after which the mixture was centrifuged to remove insoluble components (residue). The supernatant was adjusted to pH 4 and added to ammonium sulphate solution to precipitate the proteins. The isolated protein was washed thrice with water and freeze-dried to obtain keratin protein powder. Freeze-dried regenerated keratin was redissolved in ammonium hydroxide, after which ammonium thioglycolate was added, and the reaction was left to proceed for 30 minutes. Then, the pH was adjusted to pH 4 to precipitate the proteins, centrifuged at 5000 rpm for 5 minutes, and the pellet was washed thrice with water and freeze-dried to obtain a keratin protein isolate.

### (B) Amyloid Fibril Formation

Feather keratin amyloid fibrils were prepared by heating 2.5 wt. % keratin isolate solutions in 10% (v/v) acetic acid with 10 mM tris(2-carboxyethyl)phosphine hydrochloride (TCEP) at 90° C. for 5 hours. After fibrillization, amyloid fibril solutions were centrifuged at 10,000 rpm for 20 minutes to remove any aggregates.

### (C) Membrane Fabrication

To fabricate the membranes, feather keratin amyloid fibrils were mixed with a crosslinking agent (glyoxal was used in this example), and a dopant (mercaptosuccinic acid (MSA) was used in this example), cast onto a polystyrene surface, and allowed to dry. Membranes were then heat treated at 150° C. for 50 minutes to facilitate crosslinking, after which they were immersed in a peracetic acid solution and incubated at 37° C. for 5 hours. Membranes were then immersed in 0.5 M sulfuric acid (H₂SO₄) to fully protonate the sulfonic acid groups, washed with distilled water, and stored in water before further use.

Membrane samples without the addition of a dopant MSA (neat keratin) were also prepared accordingly without the addition of any dopant.

### (D) In-Situ Fuel Cell Measurements

Membranes were acidified in 0.5 M sulfuric acid before assembly and thoroughly washed with milli-Q water. Wet membranes were placed inside a 1 cm² cell between two platinum-coated commercial gas diffusion electrodes (GDE's) (JM ELE0244, Johnson Matthey, United Kingdom) with a nominal loading of 0.4 mg Pt/cm². The gas diffusion layer (GDL) compression was set to 25±1%. The cells were directly tested after assembly to prevent drying of the membranes. Fuel cell tests were performed using a custom fuel cell test bench at pounds per square inch (PSI). Laboratory Virtual Instrument Engineering Workbench (LabVIEW) was used to control and monitor the cell voltage, flow rates, and temperatures. A constant flow rate of 0.4 In/min was used for all gasses. An SP-300 potentiostat (Biologic systems, USA) was used for all electrochemical measurements. The cell temperature was controlled by two 100 W heating cartridges in the endplates.

Polarization curves were recorded 1) potentiometrically from open circuit voltage (OCV) to 0.1 V with a sweep rate of 10 mV/s or 2) galvanostatically by holding the current for 60 seconds and increasing stepwise. Measurements were performed at 100% relative humidity (RH) and a pressure of 1.5 bar (150 000 Pascal).

Hydrogen crossover currents were measured using staircase voltammetry. The potentiometric measurements were performed from 0.7 V to 0.2 V in steps of 0.1 V. Each step was held for 60 seconds and the average stable current of each step was plotted as a function of cell voltage. All measurements were performed with hydrogen (H₂) on the counter electrode (CE) and nitrogen (N₂) on the working electrode (WE) at a flow of 0.4 ln/min, 100% RH and a pressure of 1 bar (100 000 Pa).

### (E) Fuel Cell Assembly

The commercial Flex-Stak Electrochemical cell (Fuel Cell Store, USA) was used to test the keratin membranes. Wet membranes were placed between two platinum-coated carbon paper electrodes of 0.5 mg/cm² (Fuel Cell Store. USA) to form the membrane electrode assembly. Pure hydrogen gas and air were supplied at the anode and cathode, respectively. The metal tabs were connected to a mini DC/DC (LiPower, Sparkfun) converter with an output of 3.3 V.

### (F) Transistor Fabrication

Field-effect transistor devices were fabricated by depositing gold metal electrodes on top of hafnium oxide (HfO₂) (50 num) coated silicon substrate. Substrates were first cleaned by ultrasonication in acetone, isopropyl alcohol and deionized water for 10 minutes and dried using nitrogen gas. Metal electrodes were then deposited using electron-beam evaporation. An adhesion layer of chromium (10 nm) was deposited first followed by deposition of 100 nm gold on top of hafnium oxide through a shadow mask. The device has a channel length of 100 μm and width of 1 mm. 1 μL of amyloid fibril mixed with glyoxal and MSA was deposited onto the device and air-dried. The substrate was then cured at 150° C. for 50 minutes and immersed in peracetic acid solution at 37° C. for 5 hours. The device was then immersed in 0.5 M sulfuric acid for 1 hour and then rinsed thoroughly with water.

### Example 2: Protein Characterization

### (A) Sodium Dodecyl Sulfate Polyacrylamide Gel Electrophoresis (SDS-PAGE)

Freeze-dried protein isolates were prepared at 2 mg/ml dissolved in 8 M urea. 10 μL Laemmli buffer was added to 10 μL protein solution. To reduce the disulfide bonds, an additional 1 μL of 1 M tris(2-carboxyethyl)phosphine hydrochloride (TCEP) was added. SDS-PAGE was performed with a homogenous 12% gel using molecular weight (MW) markers 2 kDa to 250 kDa at 150 V for 65 minutes. The gel was then fixed with methanol/ethanol/acetic acid (MeOH/EtOH/AcOH) solution and subsequently stained with Coomassie Blue Silver Staining buffer.

### (B) Amino Acid Analysis

10 μL of feather keratin protein solutions were oxidized with 500 μL performic acid (9:1 formic acid:hydrogen peroxide) at 0° C. for 18 hours. 100 μL of hydrobromic acid was added to the solution, after which solvents were removed by speed vacuum. Samples were then hydrolyzed in 500 μL of 6 M hydrochloric acid (HCl) solution with 0.5% phenol under vacuum at 110° C. for 24 hours. Solvents were removed by speed vacuum, after which hydrolysates were washed twice with water and kept at −20° C. prior to the analysis. Composition analysis was performed with an amino acid analyzer (Sykam). The initial cysteine concentration was calculated using a conversion factor of 95% of cysteine into cysteic acid.

### (C) Atomic Force Microscopy (AFM)

20 μL of fivefold-diluted amyloid fibril solution was deposited on a freshly cleaved mica surface and incubated for 3 minutes. The surface was then washed with Milli-Q water and dried under nitrogen. Imaging with the Atomic Force Microscope (AFM) (NX10, Park Systems) was performed in non-contact mode with a scan rate of 1 Hz and scan size of 10 μm by 10 μm.

### (D) Transmission Electron Microscopy (TEM)

Amyloid fibrils were diluted to 0.2 wt. %, prepared on a copper grid and stained with uranyl acetate before imaging using transmission electron microscopy (TFS Morgagni 268) with an operating voltage of 100 kV.

### (E) Fibrillization Tracking

To determine the optimal heat treatment duration for amyloid fibril formation, 100 μL of protein solutions sampled at different heating time intervals were diluted twice with water, after which thiazole orange was added to a final concentration of 10 μM. The solutions were excited with a wavelength of 421 nm and the fluorescence emission was measured at a wavelength of 455 nm (Tecan Spark). Fluorescent intensities were normalized to the intensities measured at 0 hour.

### Example 3: Membrane Characterization

### (A) Fourier Transform Infrared Spectroscopy (FTIR)

Membranes before and after oxidation were oven dried and scanned over a range from 4000 to 600 cm⁻¹ (Varian 640) with a resolution of 2 cm⁻¹.

### (B) Raman Spectroscopy

To obtain the Raman spectrum of the membranes, samples were placed on a gold-coated glass slide (Platypus Technologies, LLC). The sample surface was focused through a 100× objective lens, after which a laser of 785 nm wavelength was applied. The spectrum was obtained with a Raman spectrometer (Horiba LabRAM HR Evolution UV-VIS-NIR) with a range from 400 to 2850 cm⁻¹ with 5 accumulations of 10 seconds acquisition time each.

### (C) Thermogravimetric Analysis (TCA)

The TGA experiment was carried out using a Mettler Toledo TGA/DSC 3+/HT with a total flowrate of 150 ml/minute of nitrogen. The temperature was increased from 25° C. to 900° C. at a rate of 10° C./min.

### (D) Scanning Electron Microscopy (SEM)

Supercritical CO₂-dried keratin membranes were fractured and mounted onto an SEM stub with conductive carbon cement. Membranes were imaged with an accelerating voltage of 2 kV (Zeiss LEO 1530) (TFS Magellan 400).

### (E) Mechanical Properties

The mechanical properties of the membranes were evaluated by tensile testing (Zwick Z010) with an applied stress of 10 kN and strain rate of 5 mm/minute. Young's moduli of the membranes were determined by the slope of the plotted stress-strain curves before the yield point. The membranes were further analyzed with dynamic mechanical analysis (MCR 702e. Anton Paar) to assess their performance with temperature. Samples of width 10 mm and 70 mm thickness were mounted between two tensile clamps at a fixed distance of 2 mm and enclosed in a chamber containing a wet cloth for humidity. After temperature equilibration, samples were tested at a fixed frequency of 1 Hz and 70 kPa stress with increasing temperature from 25° C. to 60° C.

### (F) X-Ray Diffraction (XRD)

X-ray diffractograms of the materials were collected by using a PANalytical Empyrean X-ray powder diffractometer equipped with an X'Celerator Scientific ultrafast line detector and Bragg-Brentano high-density incident beam optics using Cu Kα radiation (45 kV and 40 mA). The 2θ range was 4°-70°, the step size was 0.016° and each measurement lasted 1 hour.

### (G) X-ray Photoelectron Spectroscopy (XPS)

Supercritical CO₂ dried membranes were analyzed for their surface elemental composition using XPS (ESCALAB™ Xi+, ThermoFisher USA). Samples were placed in an analysis chamber evacuated to 8×10⁻¹⁰ Pa, to which samples were irradiated with Al kα (hv=1486.6 eV) and an applied voltage of 1 V.

### (H) Ion-Exchange Capacity (IEC) and Water Uptake (Q)

Membranes were protonated in 0.5 M sulfuric acid (H₂SO₄) overnight and rinsed thoroughly with MilliQ water and soaked in MilliQ water to remove residual H₂SO₄. Membranes were then soaked in 1 M potassium chloride (KCl) overnight and titrated with 0.05 M potassium hydroxide (KOH) until the pH reached 7. The membranes were then washed with water, weighed, and dried. The ion exchange capacity (IEC) and water uptake (Q) were calculated based on the following equation:

$$IEC = \frac{n ( {H}^{+} )}{{m}_{0}} where \qquad ( 1 )$$

$$n ( {H}^{+} ) = {c}_{KOH} \cdot {V}_{KOH} and \qquad ( 2 )$$

$${m}_{0} = {m}_{0 , {K}^{+}} - 38.1 g / mol \cdot {c}_{KOH} \cdot {V}_{KOH} \qquad ( 3 )$$

The latter correction of the membrane dry mass is necessary to account for the difference in molar mass of K⁺ and H⁺ as counter-ions.

$$Water uptake ( Q ) = \frac{{m}_{wet} - {m}_{0}}{{m}_{0}} \cdot 100 \% \qquad ( 4 )$$

### (1) Methanol Permeability

The permeability of methanol was assessed by determining the concentration of methanol diffused through the membrane clamped between two chambers—one containing 5 M methanol and the other containing pure water. The methanol permeability (P) was calculated based on the following equation:

$$P = \frac{1}{{C}_{a}} ( \frac{Δ {C}_{b} ( t )}{Δ t} ) ( \frac{L \cdot {V}_{b}}{A} ) \qquad ( 5 )$$

where P is the membrane diffusion permeability for methanol, Cₐ is the methanol concentration in the feed chamber, ΔC_{b}(t)/Δt is the methanol molar concentration in the permeate chamber as a function of time, V_{b} is the volume of each diffusion reservoir, A is the membrane area, and L is the membrane thickness.

### (J) Kelvin Probe Force Microscopy (KPFM)

KPFM was used to probe the presence of anionic functional groups on the membrane after modification. 100 μL of amyloid fibril mixed with glyoxal and MSA was deposited on a bare freshly cleaved highly ordered pyrolytic graphite (HOPG) substrate and incubated for 10 minutes for protein adsorption, after which the excess solution was removed, and the substrate was air-dried. The substrate was then cured at 150° C. for 20 minutes and immersed in peracetic acid solution at 37° C. for 5 hours. The substrate was then immersed in 0.5 M sulfuric acid for 1 hour, rinsed thoroughly with water, and mounted onto a steel disc with carbon tape.

### Example 4: Membrane Electrochemical Characterization

### (A) Electrochemical Measurements

Through plane conductivity measurements were performed with electrochemical impedance spectroscopy (EIS) using the SP-100 potentiostat (Biologic systems. USA). Wet membranes were placed in between two circular platinum electrodes with a diameter of 15 mm. Potentiostatic electrochemical impedance spectroscopy (PEIS) was used to measure the resistance as a function of frequency from 1 Mhz to 100 Hz with a 10 mV perturbation. The high frequency intercept was used to calculate the membrane resistance. Membrane samples without the addition of MSA (neat keratin) were measured from 7 MHz to 100 Hz. Proton conductivity (a) was calculated using equation (6):

$$σ = \frac{L}{R \cdot A} \qquad ( 6 )$$

where L is the thickness of the membrane, is the resistance of the membrane, and A is the contact area of the electrodes.

### (B) Protonic (H⁺) Field Effect Transistor (FET Characterization

Output and transfer characteristic of the transistor devices were recorded using the Keithley 4200A-SCS Parameter Analyzer. Devices were measured at a relative humidity level of 95% in a custom-built dry box chamber wherein relative humidity was varied using humidifier (BioAire Lifestyle) and continuously monitored using a digital hygrometer (RS PRO RS-91, ±3% RH Accuracy, 100% RH Max). The maximum source to drain voltage was restricted to 1.5 volts (thermoneutral voltage of water≈1.25 V at 25° C.) to prevent electrolysis of water.

The theoretical proton charge carrier density under different applied gate voltages was calculated using the following equation:

$${n}_{{H}^{+}} = {n}_{{H}^{+}}^{0} - \frac{{V}_{GS} \cdot {C}_{GS}}{e \cdot t} \qquad ( 7 )$$

where,

$${n}_{{H}^{+}}^{0}$$

is the charge density at zero gate voltage, V_{GS} is the gate voltage, C_{GS} is the gate capacitance. e is the proton charge, and t is the membrane thickness.

The conductivity of the keratin membrane was calculated from the slope of the I_{DS}-V_{DS} curves at different V_{GS}, where I_{DS} is the current at drain-source, and V_{DS} refers to voltage at drain-source. The plot of the conductivity as a function of the gate voltage was linear fitted with the following equation to obtain the proton mobility:

$${μ}_{{H}^{+}} = \pm \frac{\partial σ}{\partial {V}_{GS}} \cdot \frac{t}{{C}_{GS}} \qquad ( 8 )$$

where

$$\frac{\partial σ}{\partial {V}_{GS}}$$

is the slope of the linear fit, t is the thickness of the keratin membrane, and C_{GS} is the gate capacitance per unit area.

### Example 5: Protein Extraction and Isolation

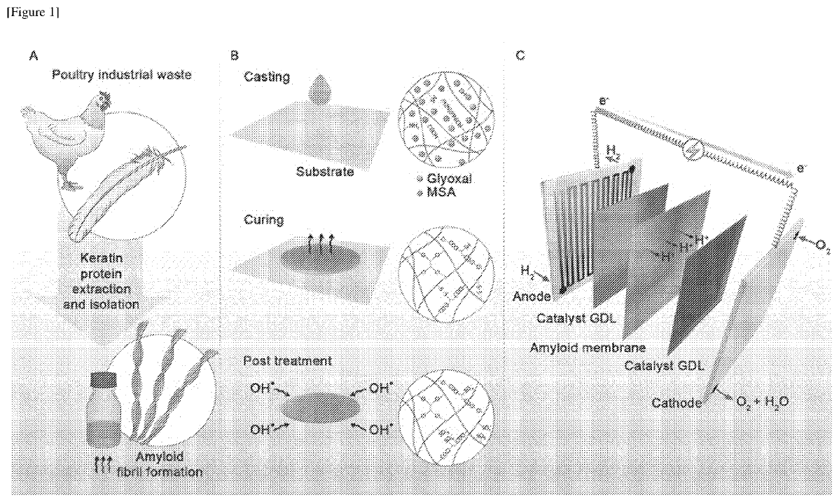

An overview of the valorization of keratin for fuel cell applications is depicted in FIG. 1, where (A) illustrates extraction, isolation, and amyloid fibril formation of feather keratin; (B) shows membrane fabrication through solvent casting, thermal curing, and post treatment; and (C) demonstrates application of keratin membrane into a fuel cell assembly.

In (A), poultry industrial waste (such as chicken feathers) undergoes keratin protein extraction and isolation to obtain keratin protein isolate. Keratin protein isolate undergoes fibrillization to form keratin amyloid fibrils. Keratin amyloid fibril solutions are obtained. After which the keratin amyloid fibril solutions were mixed with a crosslinker (e.g. glyoxal) and dopant (e.g. mercaptosuccinic acid, MSA) and cast onto a substrate to produce a free-standing membrane. The membrane was then heat cured (to facilitate crosslinking) followed by a post-oxidative treatment to produce a modified membrane with imparted proton conductive properties (as shown in (B) of FIG. 1), which was then assembled into a fuel cell (as shown in (C) of FIG. 1). The film is cured to induce crosslinking, after which it is immersed in a solution consisting of peroxide to induce oxidation and producing a film containing sulfonic acids with proton conductive properties. The entire fabrication process does not employ harsh and toxic reagents while also utilizing reagents used commonly in industry, keeping costs low.

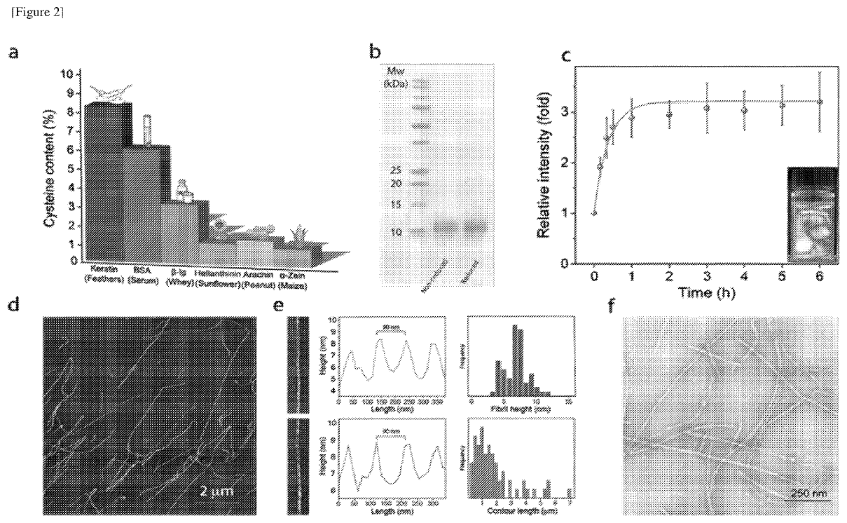

Among protein-containing industrial waste by-products, chicken feathers have the highest protein contents (about 90%), with the richest cysteine (Cys) composition (about 8%, FIG. 2 (a)). However, the extraction and isolation procedure of feather keratin on a large scale has faced challenges, notably the use of harsh toxic solvents (e.g. sodium sulfide) and reducing agents such as β-mercaptoethanol and dithiothreitol, or long isolation dialysis treatments.

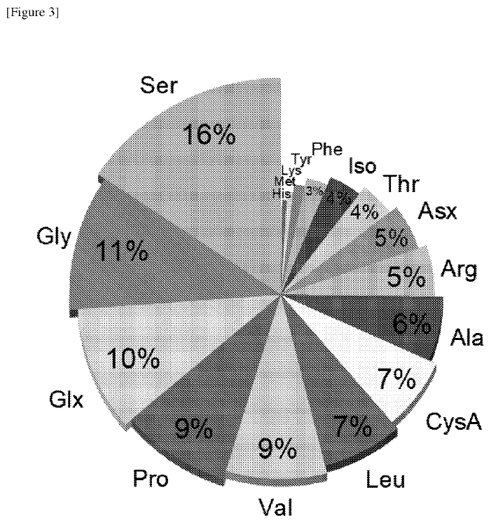

In this example, it is demonstrated that feather keratin was extracted using a basic solvent consisting of urea and thioglycolate. Urea acted as a chaotropic solvent disrupting the hydrogen bonds within the compact structure of feathers, while thioglycolate served as a reducing agent to reduce intra- and intermolecular disulfide bonds, resulting in the separation of protein inter-chains and eventually dissolution. The supernatant was then precipitated and washed to obtain a crude keratin isolate. The isolate was re-extracted in ammoniacal solution and precipitated it to obtain a pure keratin isolate, which displayed only a single protein band around 10 kDa as analyzed with electrophoresis (FIG. 2 (b)), demonstrating minimal protein degradation during the entire process. An amino acid analysis of the obtained keratin isolate revealed 7 mol % of Cys (FIG. 3), in agreement with the expected 8 mol % from the results disclosed herein. This shows that the process produced a feather keratin isolate possessing almost full solubility in dilute acid in contrast to keratin isolates obtained from other extraction procedures, which require concentrated acids. Furthermore, the process does not require dialysis, enabling scalability and fast production rates.

### Example 6: Protein Fibrillization

Within the family of keratin proteins, hair and wool keratins belong to α-keratin composed of α-helix intermediate filaments, while keratin from feathers, claws, and beaks belong to β-keratins dominated by α-sheet secondary structures.

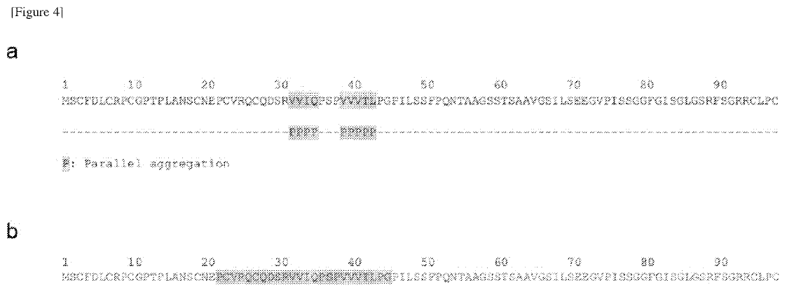

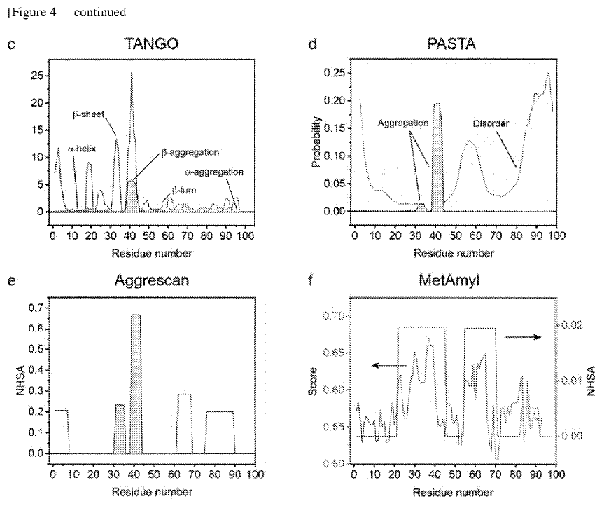

Sequence analysis of feather keratin from the UniProt database (P02450) using a variety of protein structure prediction algorithms (FIG. 4) indicated that feather keratin could be assembled into amyloid fibrils. The structure of feather keratin has been reported to consist of a central region with predominantly hydrophobic residues adopting a β-sheet conformation, with cysteine (Cys) residues primarily located at the N- and C-termini. The highest propensity for β-sheet aggregation was predicted to occur between residues Val32-Leu43, which lie within this central region and have also been signalled out as a hotspot for amyloidogenic aggregation. Thus, in this example, the inventors postulated that feather keratin could be self-assembled into amyloid fibrils, which was verified by the facile self-assembly of keratin monomers into amyloid fibrils after heat treatment at 90° C. under acidic conditions.

Using thiazole orange as a molecular probe for amyloid fibrils, the fibrillization process proceeded relatively quickly and reached a plateau after only 2 hours, exhibiting a faster kinetic than that of animal-derived β-lactoglobulin or plant-derived proteins (FIG. 2 (c)). Solutions of feather keratin amyloid fibrils also exhibited birefringence under cross-polarized light, corroborating the presence of amyloid fibrils (inset of FIG. 2 (c)), which were found to be several micrometers in length after 5 hours as seen from Atomic Force Microscopy (AFM) and Transmission Electron Microscopy (TEM) images (FIG. 2 (d) and FIG. 2 (f)). The nanofibrils exhibited periodic pitches of 90 nm along the fibril axis with an average height of about 7 nm (FIG. 2 (e)), as expected in most amyloid fibrils.

### Example 7: Membrane Fabrication

Feather keratin amyloid fibrils were mixed with glyoxal and mercaptosuccinic acid, and the resulting solution was casted to obtain a clear free-standing flexible membrane (FIG. 5 (a)), and further went through thermal curing (FIG. 5 (b)). As the inventors hypothesized that proton conduction could be imparted into the membrane upon conversion of thiol groups into sulfonic acids, the membranes were immersed in peracetic acid to oxidize thiols and disulfide bonds to sulfonic acids (FIG. 5 (c)). The final membrane is denoted as Keratin-M.

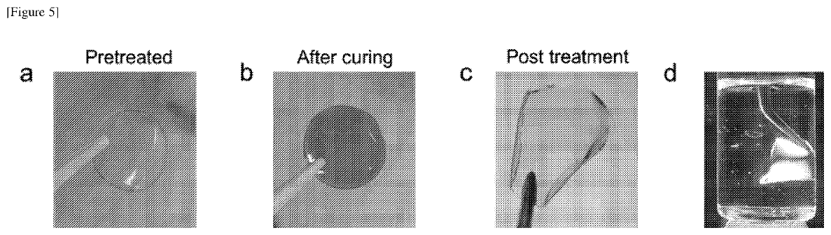

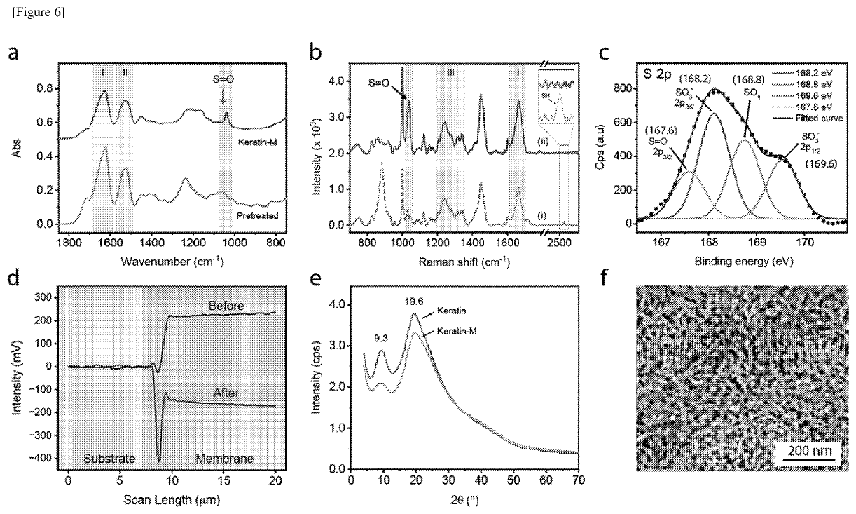

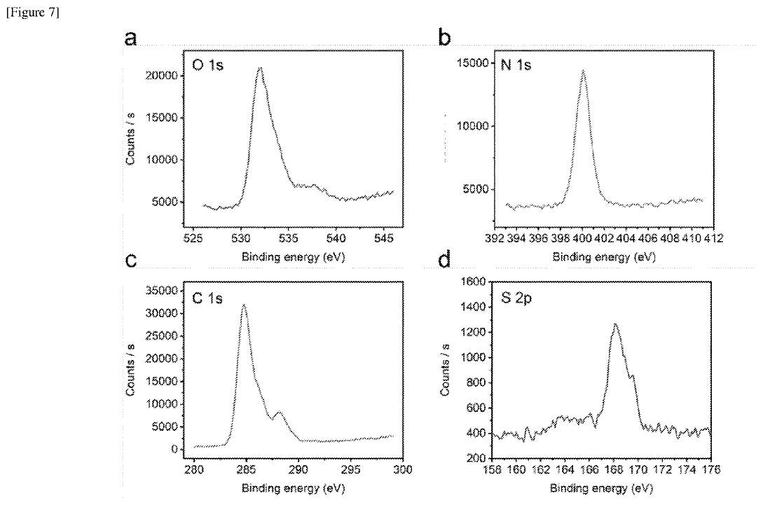

Oxidation into sulfonic acid was corroborated by both Fourier Transform Infrared Spectroscopy (FTIR) and Raman Spectroscopy, with the appearance of the S═O peak at 1040 cm⁻¹ (FIG. 6 (a)) in the FTIR spectrum, and the disappearance of the Raman shift peak at 2550 cm⁻¹ assigned to the thiol (FIG. 6 (b)). This was further supported by XPS measurements (FIG. 7), which showed the major peaks of —SO₃⁻ 2p_{3/2} and 2p_{1/2} doublets at 168.2 and 169.6 eV, respectively, after deconvolution. Peaks of sulfone and sulfate groups were also observed, possibly due to side reactions in the fabrication process (FIG. 6 (c)). As the modification of thiols into sulfonic acids confers them a negative charge, the difference in surface potential before and after modification was detected by Kelvin Probe Force Microscopy (KPFM). Before modification, the keratin casting solution deposited onto the Highly Oriented Pyrolytic Graphite (HOPG) substrate exhibited a relative positive potential, which likely arose from positively charged amino acids arginine (Arg) and lysine (Lys). Upon post oxidative treatment, the membrane exhibited a relative negative potential that may have resulted from the conversion of neutral thiols into negatively charged sulfonic acids (FIG. 6 (d) and FIG. 8).

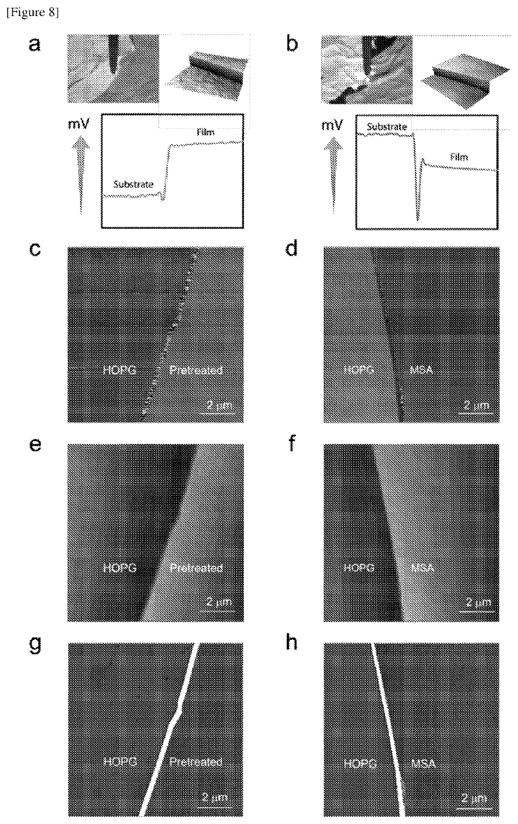

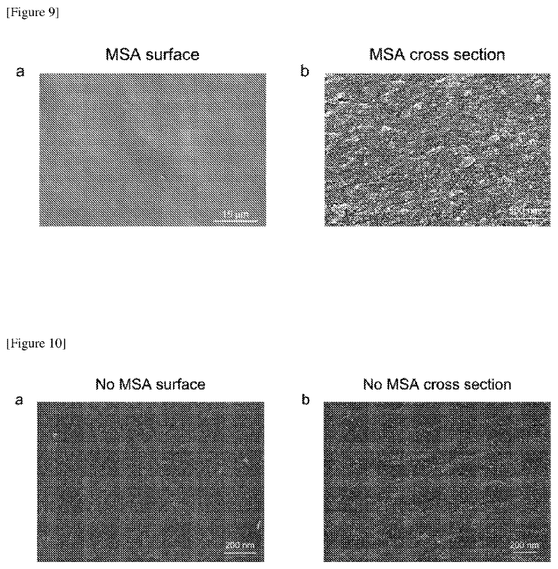

Throughout the fabrication process, the membrane maintained its shape and mechanical integrity while still possessing birefringence arising from amyloid fibrils under cross-polarized light (FIG. 5 (d)), suggesting that the amyloid fibrils were preserved without much alteration. The XRD pattern of the membranes displayed two dominant peaks at 9.3° and 19.6°, corresponding to d-spacings of 4.5 Å and 9.5 Å, respectively, that reflect the signature cross-b structure of amyloid fibrils (FIG. 6 (c)). A smooth surface was observed under SEM for keratin-M membranes (FIG. 9), whereas at higher magnification a fibrillar network morphology was identified on the surface with pore sizes of 7-20 nm (FIG. 6 (f)). In contrast, neat keratin membranes showed a denser morphology with less porosity (FIG. 10).

### Example 8: Membrane Properties

The modification of neutral thiols into negatively charged sulfonic acids was also indicated by the increase in ion exchange capacity (IEC) from 0.18 meq g⁻¹ to 0.84 meq g⁻¹ after oxidative treatment, while a further increase to 1.56 meq g⁻¹ was achieved with the addition of MSA due to the contribution of additional thiol groups (Table 1).

TABLE 1
Feather keratin membrane properties before and after treatment

| Membrane | IEC (meq g⁻¹) | Water Uptake (%) |
|---|---|---|
| Pre-treated | 0.18 ± 0.03 | 55.3 ± 6.7 |
| Neat keratin | 0.84 ± 0.05 | 131.3 ± 17 |
| Keratin-M | 1.56 ± 0.03 | 215.9 ± 31 |
| Nafion 117 | 1.10 ± 0.02 | 17.0 ± 1.2 |

The boost in IEC also resulted in a higher water uptake due to enhanced hydration and charge repulsion from the anionic sulfonic acid groups, which could possibly explain the larger pore size from keratin-M due to higher swelling compared to neat keratin membranes. Keratin-M membranes maintained good barrier properties despite the presence of nanopores, demonstrating almost no permeability to large dye molecules such as Rhodamine B (FIG. 11).

The keratin-M membranes also exhibited 3-fold less permeability to small molecules such as methanol compared to Nafion (FIG. 12), demonstrating their potential in direct methanol fuel cells in which Nafion performs less efficiently due to the high methanol permeability of the latter.

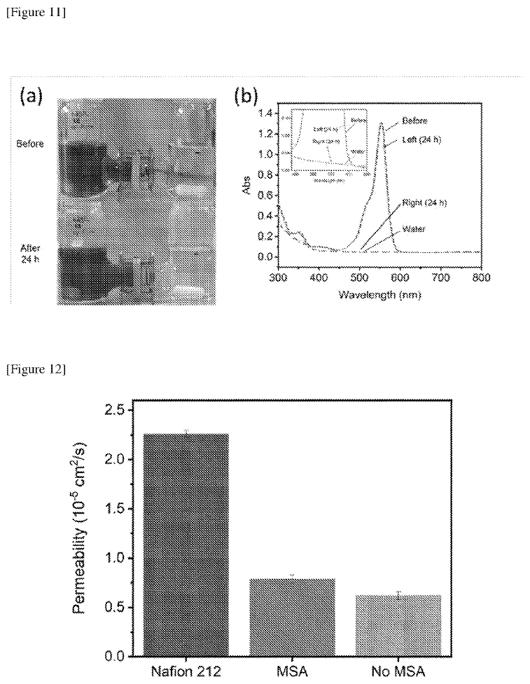

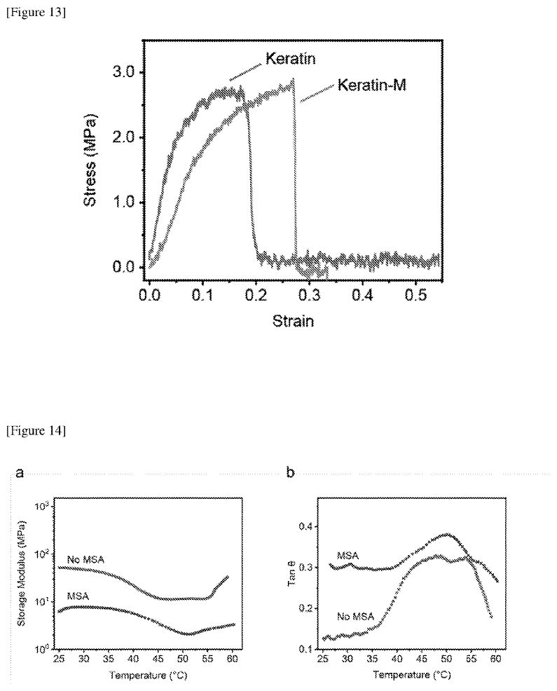

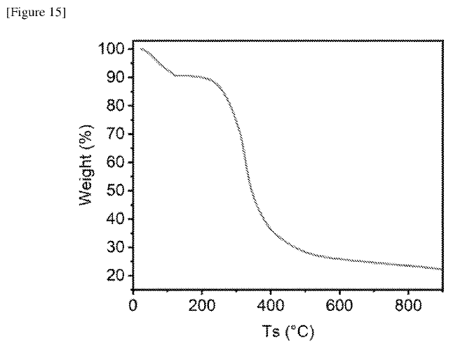

The increased swelling in keratin-M also resulted in a softer and more clastic material with a lower Young's modulus (Table 2) in contrast to neat membranes, which were stronger but more brittle (FIG. 13). Keratin membranes also showed a decrease in rigidity above 50° C. with a similar transition in the loss modulus tan 0 at 50° C., which suggests the onset of membrane softening due to membranes' glass transition temperature (FIG. 14). Supercritical CO₂-dried keratin membranes measured with TGA displayed a 10% decrease in weight until 100° C. attributed to the evaporation of water, followed by thermal degradation and deamidation with drastic weight loss (~60%) between 300° C. to 400° C. (FIG. 15).

TABLE 2
Mechanical properties of keratin membranes.

| | Young's modulus | Yield strength | Ultimate Tensile |
|---|---|---|---|
| Membrane | (MPa) | (MPa) | Strength (MPa) |
| Keratin | 35.5 | 2.35 | 3.33 |
| Keratin-M | 25.6 | 1.61 | 2.21 |

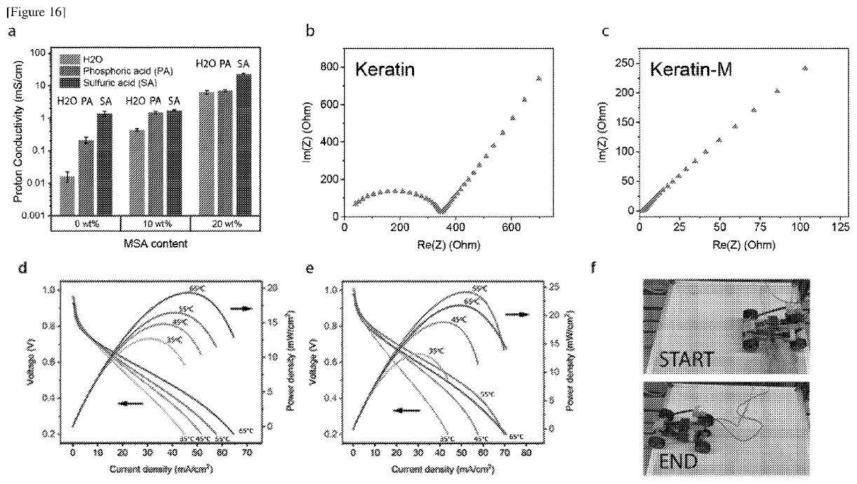

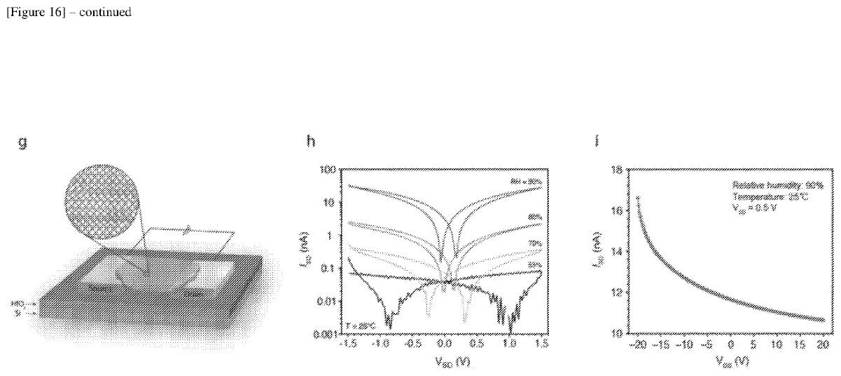

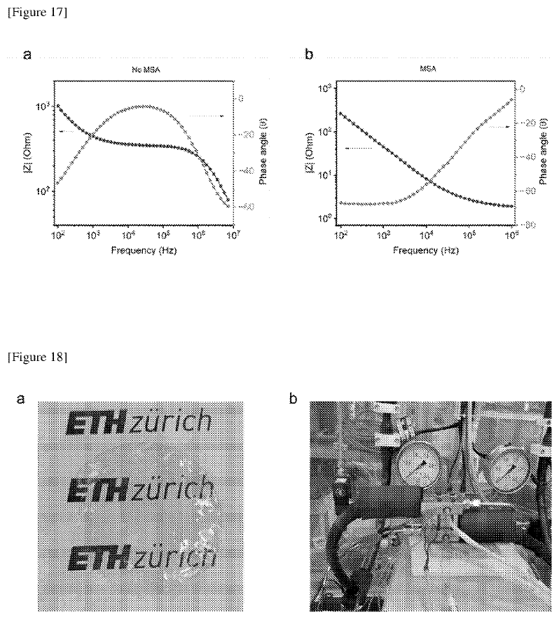

The main parameter in a fuel cell is the proton conductivity (a), which is essential for any polymer electrolyte membrane. After the successful conversion of thiols into sulfonic acids, feather keratin membranes exhibited an enhanced proton conductivity that increased with the MSA content from 0.02 mS cm⁻¹ to 6.3 mS cm⁻¹ in water (FIG. 16 (a). The appearance of the semi-circle in the Nyquist plot of neat membranes indicate a more capacitive behaviour (FIG. 16 (b)), as also evidenced in the large phase angle from the Bode plot (FIG. 17 (a)). This characteristic shifted towards a membrane with a resistor-type behaviour for keratin-M membranes, as seen from an almost absent semi-circle in the Nyquist plot (FIG. 16 (c)) and a phase angle approaching 0° at high frequencies in the Bode plot (FIG. 17 (b)). Comparing across biomaterials reported to date, the proton conductivity of keratin-M attained is lower than specifically engineered peptides, but highest among all other biopolymers (Table 3 and Table 4). But in contrast to engineered peptides, feather keratin membranes are processed from low-value waste materials with a strong potential for scaling-up; remarkably, because this comes from a waste stream intended for incineration (and CO₂ emissions), this process takes place with an overall negative carbon footprint, adding value to both sustainability and environmental friendliness. Furthermore, the proton conductivity of the membranes could still be further improved by doping with acid electrolytics, notably with sulfuric acid which provided the most significant increment to 22.8 mS cm⁻¹.

TABLE 3
Proton conductivities of natural biomaterials

| | Proton | Relative |
|---|---|---|
| | conductivity | humidity |
| Natural biomaterials | (mS/cm) | (%) |
| SRT motifs | 3.5 | 100 |
| Ampullae of Lorenzini | 1.8 | 90 |
| Chitosan | 1.5 | — |
| Carrageenan | 0.9-1.1 | — |
| Squid Reflectin A1 | 0.1 | 90 |

TABLE 4
Proton conductivities of engineered biomaterials

| | Proton | Relative | |
|---|---|---|---|
| | conductivity | humidity | |
| Engineered biomaterials | (mS/cm) | (%) | |
| Tyrosine-rich peptide | 18.6 | 90 | |
| Chimera polypeptide | 18.5 | 90 | |
| Keratin-M | 6.3 | 95 | Present invention |
| BSA film | 5.3 | 75 | |
| CNF | 1.2-1.5 | 95 | |
| Maleic chitosan | 0.7 | 75 | |

### Example 9: Fuel Cell Performance

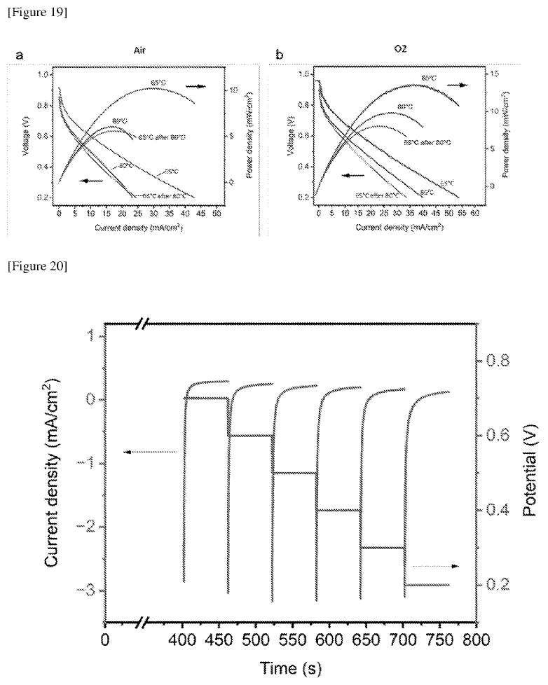

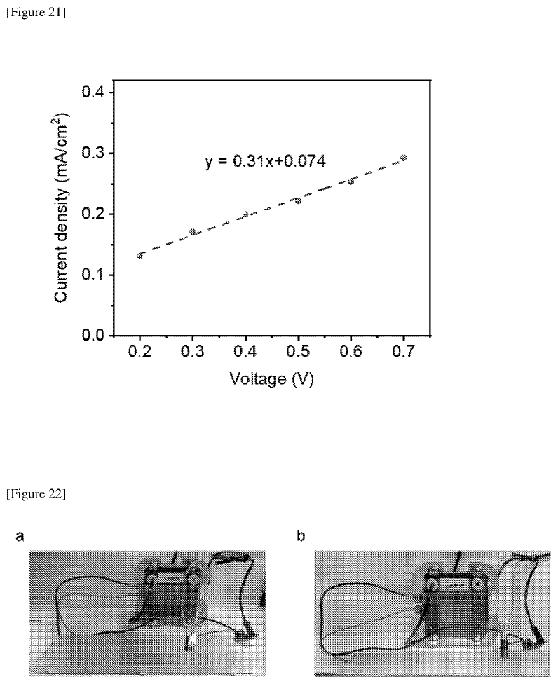

Performance of the keratin membrane as a polymer electrolyte membrane in an in-situ hydrogen fuel cell was tested (FIG. 18). The open circuit voltage (OCV) of a fuel cell has been reported to provide a useful indication of the integrity and degradative behavior of the membrane such as thinning and pinhole formation. The cell assembled with keratin-M membrane displayed an OCV between 0.95-1 V (comparable to Nafion) and polarization curves were obtained at increasing temperature. With air at the cathode, the cell generated an increased power density with temperature, reaching up to 20 mW cm² at 65° C. (FIG. 16 (d)). Similarly, the peak power of 25 mW cm⁻² was generated with pure oxygen at 55° C., after which it decreased to 21 mW cm⁻² at 65° C. (FIG. 16 (e)). This could be due to an increase in reactive oxygen species generated with increased oxygen concentration, leading to the formation of free radicals such as OH·, H·, and HOO, that have been reported to initiate membrane degradation. Further increase in temperature to 80° C. resulted in a drop in OCV to 0.85 to 0.9 V along with a decreased power density lower than that at 65° C. Moreover, the membrane performance did not recover upon reducing the temperature back to 65° C., suggesting possible irreversible membrane degradation or softening (FIG. 19). To ensure a high OCV and optimal performance, a membrane of low hydrogen permeability is desired. Utilizing staircase voltammetry with nitrogen (N₂) at the cathode (FIG. 20), the hydrogen permeability of the keratin membrane was assessed and calculated using the intercept, yielding 3.8±0.3×10⁻¹⁰ mol cm⁻² s⁻¹, (compare to Nafion—3.8×10⁻⁹ mol cm⁻² s⁻¹) while the electrical resistance obtained from the slope was approximately 3200±140 Ohm cm² (FIG. 21).

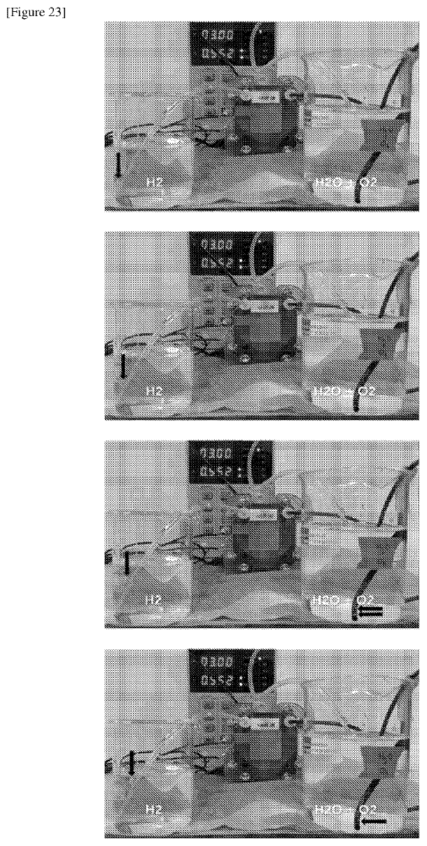

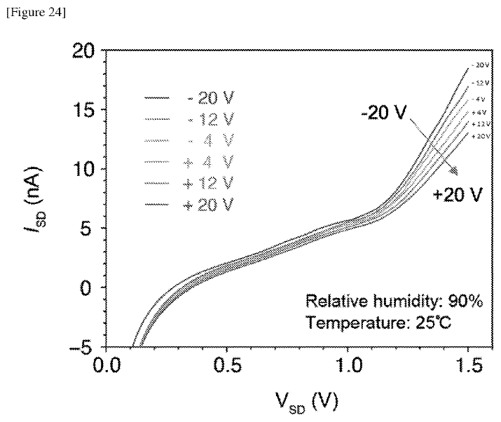

To demonstrate the applicability of the keratin-M membrane in hydrogen fuel cells, the membrane was assembled into a commercial test fuel cell setup. With hydrogen and air as the respective fuels at the anode and cathode, the cell was able to generate power to turn on both red and white LED lamps (FIG. 22). In addition, the cell was responsive to the presence of fuel, turning the LED lamp on and off with the introduction and absence of hydrogen. The fuel cell could perform mechanical work, e.g., drive a fan setup driven by a motor and also a fuel cell toy car (FIG. 16 (f)). Conversely, the keratin-M PEM membranes can be further used as an electrolyzer for the production of hydrogen and oxygen from water using electricity, as observed in the formation of bubbles at the respective outlets (FIG. 23).

### Example 10: Transistor Performance

The application of keratin-M as a solid-state film for the fabrication of protonic field-effect transistors (H⁺-FETs) was demonstrated. Devices were fabricated by casting keratin-M film between two gold electrodes pre-deposited using e-beam evaporation process on a hafnium oxide (HfO₂) gate dielectric film (FIG. 16 (g)). In H⁺-FETs, the magnitude of current is determined by the total proton charge carrier density (n_{H}_{⁺}) obtained from equation (7). When a negative gate potential is applied, proportional positive charges are induced in the channel as the conducting silicon gate and keratin-M form a capacitor with a HfO₂ dielectric between them. To create additional positive charges, protons are injected into the channel from the electrodes, thereby increasing n_{H}_{⁺}, whereas the application of positive gate voltages deplete proton density. As seen in the output (FIG. 24) and transfer characteristics of the H⁺-FET device (FIG. 16 (b)), the transistor exhibited a strong voltage-gated H⁺ conductivity in the channel. The inventors calculated n_{H}_{⁺}≈4.0×10¹⁷ cm⁻³ (V_{DS}=1.5 V and 95% RH) at V_{GS}=0 V, which increased to a 5.6×10¹⁷ cm⁻³ at V_{GS}=−5 V and decreased to t 3.5×10¹⁶ cm⁻³ at V_{GS}=+5 V, corresponding to a high on-to-off current ratio I_{HIGH}/I_{LOW} of ≈9.7. It was further determined the mobility of proton from the linear fit to the slope of the conductivity-gate voltage plot

$$( \pm \frac{\partial σ}{\partial {V}_{GS}} )$$

and obtained a mobility of μ_{H}_{⁺}≈1.54×10⁻³ cm² V⁻¹s⁻¹ using equation (SS). In addition, this gating effect was demonstrated to be reversible observed from the restoration of current upon returning to negative V, (FIG. 16 (i)), highlighting the potential of keratin-M as functional protonic transistors.

The fabrication of proton conductive membranes from feather keratin (isolated from chicken feathers) was demonstrated. The methods were green and low-cost with high scalability. Keratin membranes were successfully used in a hydrogen fuel cell to power several devices with only hydrogen and air as the fuel, and also demonstrated pronounced electrostatic gating effect in a transistor and hydrogen generation in an electrolyzer. The disclosure using industrial waste materials reprocessed into valuable materials using a sustainable process brings us one step closer towards a zero-carbon and circular economy.

### INDUSTRIAL APPLICABILITY

The present invention relates to a method of obtaining keratin protein isolate from a keratin source, comprising steps as disclosed herein. The present invention also relates to a method of obtaining keratin amyloid fibrils, comprising steps as disclosed herein. The present invention further relates to a method of fabricating a membrane for an electrochemical cell, comprising steps as disclosed herein. The keratin sources used in these methods are low-cost, easily available and valuable biological raw materials, which can be reconfigured into keratin protein isolate and may be used for highly functional materials. The disclosed methods are easy-to-perform, safe and does not pose any toxicity issues. The chemicals used in the disclosed method are also easily obtained, safe (not harsh) reagents and low-cost. The disclosed method to obtain keratin amyloid fibrils is a simple, straightforward, safe and effective process. The disclosed method for fabricating a membrane for electrochemical cell is a sustainable, green, cost-effective method, simple, straightforward, easy-to-perform and allows for expansion to large-scale applications. The membrane obtained from the disclosed methods demonstrates good performance and is widely applicable and may be used for various electrochemical applications such as in fuel cells and protonic field-effect transistors.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A method of obtaining keratin protein isolate from a keratin source, the method comprising:
(a) preparing a first mixture comprising keratin source, chaotropic solvent, and a first reducing agent;
(b) precipitating keratin protein from the first mixture;
(c) preparing a second mixture comprising the keratin protein and a second reducing agent; and
(d) precipitating keratin protein isolate from the second mixture.

2. The method of claim 1, wherein step (a) comprises heating the first mixture at a temperature of about 50° C. to about 80° C.

3. The method of claim 1, wherein step (b) comprises adjusting the pH of the first mixture to about pH 3 to about pH 5 and adding a first solution to precipitate the keratin protein.

4. The method of claim 1, further comprising freeze drying the keratin protein after step (b).

5. The method of claim 1, wherein step (c) further comprises dissolving the keratin protein in a second solution and subsequently adding the second reducing agent.

6. The method of claim 1, wherein step (d) comprises adjusting the pH of the second mixture to about pH 3 to about pH 5 to precipitate the keratin protein isolate.

7. The method of claim 1, further comprising freeze drying the keratin protein isolate after step (d).

8. The method of claim 1, wherein the keratin source is selected from the group consisting of hair, nails, feathers, claws, beaks, horns, hoofs, and wools from human, birds and/or animals.

9. The method of claim 1, wherein the chaotropic solvent is selected from the group consisting of urea, thiourea, guanidinium chloride, lithium perchlorate, lithium acetate, and sodium dodecyl sulfate.

10. The method of claim 3, wherein the first solution comprises a salt or base independently selected from the group consisting of ammonium sulfate, sodium hydroxide, potassium hydroxide, ammonium hydroxide, and tris(hydroxymethyl)aminomethane.

11. A method of obtaining keratin amyloid fibrils, the method comprising:
(i) obtaining keratin protein isolate according to claim 1;
(ii) preparing a third mixture comprising keratin protein isolate, acid solution, and a third reducing agent; and
(iii) subjecting the third mixture to heat treatment to obtain keratin amyloid fibrils.

12. The method of claim 11, the heat treatment comprises heating the third mixture at a temperature of about 80° C. to about 100° C., and/or wherein the heat treatment is performed for about 2 hours to about 24 hours; and/or wherein the acid solution is selected from the group consisting of hydrochloric acid, acetic acid, formic acid, propionic acid, tartaric acid, malonic acid, oxalic acid, pyruvic acid, and citric acid.

13. (canceled)

14. (canceled)

15. A method of fabricating a membrane for an electrochemical cell, the method comprising:
(A) forming a film by combining keratin amyloid fibrils obtained from claim 11, a crosslinking agent, and a dopant;
(B) heat treating the film to induce crosslinking; and
(C) subjecting the crosslinked film to oxidation and protonation to form the membrane.

16. The method of claim 15, wherein step (B) comprises heating the film at a temperature of about 150° C. to about 180° C. and/or wherein step (B) is performed for about 30 minutes to about 60 minutes.

17. (canceled)

18. The method of claim 15, wherein step (C) comprises immersing the crosslinked film in an oxidizing agent selected from the group consisting of consisting of peroxide, peracetic acid, peroxy acids including performic acid, peracetic acid, peroxymonosulfuric acid, and peroxymonophosphoric acid; and/or wherein step (C) comprises immersing the crosslinked film in an acid selected from the group consisting of organic acids, hydrochloric acid, sulfuric acid, sulfonic acids, phosphoric acid, nitric acid, to protonate the membrane.

19. (canceled)

20. The method of claim 15, wherein the crosslinking agent is selected from the group consisting of formaldehyde, glyoxal, malondialdehyde, succindialdehyde, and glutaraldehyde.

21. The method of claim 15, wherein the dopant is selected from the group consisting of mercaptosuccinic acid, mercaptoacetic acid, and 3-mercaptopropionic acid.

22. The method of claim 1, wherein the first and second reducing agent are independently selected from the group consisting of ammonium thioglycolate, thiols, dithiothreitol, β-mercaptoethanol, 2-mercaptoethanol, dithiothreitol, 3-mercapto-1,2-propandiol, thioglycolic acid, tris(2-carboxyethyl)phosphine, tris(hydroxypropyl)phosphine, and tris(hydroxymethyl)phosphine.

23. The method of claim 11, wherein the third reducing agent is selected from the group consisting of ammonium thioglycolate, thiols, dithiothreitol, β-mercaptoethanol, 2-mercaptoethanol, dithiothreitol, 3-mercapto-1,2-propandiol, thioglycolic acid, tris(2-carboxyethyl)phosphine, tris(hydroxypropyl)phosphine, and tris(hydroxymethyl)phosphine.

24. The method of claim 5, wherein the second solution comprises a salt or base independently selected from the group consisting of ammonium sulfate, sodium hydroxide, potassium hydroxide, ammonium hydroxide, and tris(hydroxymethyl)aminomethane.
